(19) United States
(12) Patent Application Publication
(10) Pub. No.: US 2026/0000306 A1
(43) Pub. Date: Jan. 1, 2026

(54) ON-DEMAND BLOOD PRESSURE MEASUREMENT DEVICES AND METHODS

(71) Applicant: Apple Inc., Cupertino, CA (US)

(72) Inventors: Vivek Venugopal, Santa Clara, CA (US); Albert E. Cerussi, San Jose, CA (US); Joseph M. Schmitt, Santa Clara, CA (US); Pranay Jain, Mountain View, CA (US); Marduke Yousefpor, San Jose, CA (US); Sohrab Eslami, Austin, TX (US)

(21) Appl. No.: 19/251,282

(22) Filed: Jun. 26, 2025

Related U.S. Application Data
(60) Provisional application No. 63/666,057, filed on Jun. 28, 2024.

(51) Int. Cl. A61B 5/022; A61B 5/00; A61B 5/021; A61B 5/026; A61B 5/0295

(52) U.S. Cl. CPC A61B 5/022; A61B 5/02108; A61B 5/0261; A61B 5/0295; A61B 5/7253

(57) ABSTRACT

Embodiments are directed to systems and methods for determining blood pressure (e.g., diastolic and systolic pressure) of a user. A pressure applied by a skin region of a user and an optical cardiac signal are simultaneously measured. The optical cardiac signal, which may correspond to a measured skin region blood volume, is analyzed to identify a series of pulse waves, which in some instances may be normalized. A series of values of a metric associated with the morphology of the pulse waves is determined. The metric values are associated with corresponding pressure values. The metric values and pressure values are used to determine diastolic pressure and systolic pressure.

### CROSS-REFERENCE TO RELATED APPLICATIONS

This application is a nonprovisional and claims the benefit under 35 U.S.C. § 119 (e) of U.S. Provisional Patent Application No. 63/666,057, filed Jun. 28, 2024, the contents of which are incorporated herein by reference as if fully disclosed herein.

### FIELD

The described embodiments relate generally to systems and methods for determining blood pressure using pressure and blood volume signals. More specifically, the blood pressure may be determined using morphological analysis of pulse waves in the blood volume signal.

### BACKGROUND

Blood pressure (BP) measurements provide valuable health data that may be used to help a user better understand their health and wellness. Outside of a clinical setting, a user may take BP readings using an at-home, inflatable arm cuff, which may allow the user to perform BP measurements on-demand. While these cuff-based devices may allow for more frequent BP measurements, such devices may not provide a convenient or practical solution for many users. For example, certain cuff-based devices may be loud, cumbersome, and/or uncomfortable to use. Accordingly, it may be desirable to provide a device that may perform BP measurements without needing to rely on an inflatable cuff.

### SUMMARY

Embodiments described herein are directed to systems and methods for estimating a user's blood pressure using measured pressure and cardiac signals. Some embodiments are directed to a method for determining blood pressure, comprising measuring a pressure signal corresponding to a pressure applied by a skin region of a user over a measurement period; measuring a blood volume signal from the skin region during the measurement period; and identifying a series of pulse waves within the blood volume signal. The method further includes determining a series of metric values, wherein each metric value is associated with a morphology of a respective pulse wave. The method generates a series of data points, where each data point comprises a pressure value from the pressure signal and a corresponding metric value from the series of metric values. The method further determines a diastolic pressure and/or a systolic pressure based on the series of data points.

Embodiments are also directed to a method for determining blood pressure, where the method includes measuring a pressure applied by the skin region of a user over a measurement period and simultaneously measuring a skin region blood volume over the measurement period using an optical sensor. The method includes identifying a series of pulse waves within the measured blood volume. The method generates a set of data points, each data point comprising a pressure value of the determined skin region pressure and a value of a metric associated with a morphology of each pulse wave. The diastolic and systolic pressures are determined from the set of data points, and at least one of the systolic and diastolic pressures are presented.

Embodiments are also directed to a device that includes a set of sensors configured to collect data associated with blood pressure, a memory, and one or more processors operatively coupled to the memory. The one or more processors are configured to execute instructions causing the one or more processors to measure a pressure signal corresponding to a pressure applied by a skin region of a user over a measurement period. The instructions further cause the one or more processors to measure a blood volume signal from the skin region during the measurement period, identify a series of pulse waves within the blood volume signal. The device processor(s) determine a series of metric values, wherein each of the metric values is associated with a morphology of a respective pulse wave. The device processor(s) further generate a series of data points, each data point comprising a pressure value from the pressure signal and a corresponding metric value. Based on the series of data points, the device processor(s) determine the diastolic and systolic pressures.

In addition to the example aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIG. 1A depicts exemplary components of a device that can be used to determine the blood pressure of a user, as described herein.

FIGS. 1B and 1C depict front views and FIG. 1D depicts a rear view, respectively, of a variation of the device of FIG. 1A.

FIG. 2A depicts a cross-sectional side view of a sensing assembly that may be used by the devices described herein to perform a blood pressure measurement.

FIGS. 2B-2C depict top views of a sensing assembly that includes an optical sensor that may be used to perform a blood pressure measurement.

FIG. 3A depicts a graph that includes an example cardiac signal that may be captured as part of a blood pressure measurement as described herein.

FIG. 3B depicts a graph that includes an example pressure signal that may be captured as part of a blood pressure measurement as described, as well as a pressure profile for assisting in obtaining the pressure signal. FIG. 3C depicts a graph that includes another example of a pressure profile as described herein.

FIG. 4 depicts a variation of a process for determining blood pressure as described herein.

FIG. 5A depicts examples of normalized pulse waves used to determine AUC values.

FIG. 5B depicts a graph of example data points used to determine blood pressure based on AUC and pressure values.

FIG. 6A depicts an example ECG signal and corresponding pulse wave used to determine a PAT value.

FIG. 6B depicts a graph of example data points used to determine blood pressure based on PAT and pressure values.

FIGS. 7A and 7B depict top views of a sensing assembly that includes a fingerprint sensor that may facilitate finger placement during a blood pressure measurement.

FIGS. 8A and 8B depict top views of a sensing assembly that includes an optical sensor that is operable to measure different skin regions of a user during a blood pressure measurement.

FIG. 9 depicts a process for performing a blood pressure measurement using a target skin location.

FIG. 10A depicts a process for determining a target skin location as described herein.

FIG. 10B shows a graph of a blood volume signals that includes multiple pulse waves corresponding to different finger positions. FIG. 10C shows a chart of measured optical signal values as a function of finger position.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments disclosed herein are directed to systems and methods for performing on-demand blood pressure measurements, which may be used to measure one or more of a user's mean arterial pressure, diastolic blood pressure, or systolic blood pressure. During a measurement, a user may press a region of skin (e.g., a portion of a finger, such as a fingertip, a wrist, a forehead, a temple, or the like) against a sensing assembly, and may apply a varying pressure to the sensing assembly over a measurement period. The sensing assembly is configured to measure both i) a pressure applied to the sensing assembly (e.g., by the user's skin) during the measurement period and ii) a cardiac signal (e.g., a blood volume signal or a blood flow signal) through the user's skin during the measurement period. For example, the sensing assembly includes a force sensor or pressure sensor for measuring the applied pressure and an optical sensor for measuring the cardiac signal. The sensing assembly may be included as part of an electronic device, such as a smartwatch, smart telephone, tablet, laptop computer, or the like.

The devices and methods are described herein as using a finger as an example of a skin region that is measured as part of a blood pressure measurement. It should be appreciated that the devices and methods described herein may measure other skin regions of a user, such as a portion of an arm (e.g., a wrist) or a leg of a user. For example, a user may use a hand to press the sensing assembly of the device in contact with a skin region of another portion of the user's body. By pressing the sensing assembly against the skin region, the skin region may apply a reciprocal force to the sensing assembly that may be measured by the sensing assembly. In one example, the sensing assembly may be incorporated into a portion of a smartwatch, and the user may press the smartwatch (and thereby the sensing assembly) against their wrist. The sensing assembly may measure a pressure between the user's wrist and the sensing assembly and may also measure a cardiac signal from the user's wrist, and may analyze these signals to determine a user's blood pressure.

In some examples, the pressure and blood volume measurements may be acquired by sensing components associated with a display of the device. For example, the device may include a force-sensitive display capable of measuring an applied force (e.g., applied to the display or a portion thereof). The device may further determine the surface area of contact between the finger of the user and the display, and may use the determined surface area and measured force to further determine the applied pressure. The device may determine the contact surface area using any of a variety of established methods, such as by using an imagining technique (e.g., imaging sensors), by using touch-sensitive components of the display, and/or by using other established methods for determining the contact area. The display may also include optical sensing components positioned beneath the surface of the display that are capable of measure finger blood volume as described herein.

The measured cardiac signal may be used to identify a series of pulse waves. Each pulse wave corresponds to a blood pressure wave transmitted through the vasculature of the user as a result of a cardiac contraction (e.g., a heartbeat). Each pulse wave may be associated with a pressure value that is selected using the pressure measured by the sensing assembly, and may be analyzed to determine one or more metric values associated with a morphology of that pulse wave. Accordingly, a series of data points may be generated for the measurement period, where each data point includes i) a pressure value associated with a respective pulse wave and ii) the one or more metrics values associated with the respective pulse wave. The series of data points may be analyzed to determine a mean arterial pressure, a diastolic blood pressure and/or a systolic blood pressure of a user.

As a user presses their skin against the sensing assembly, the applied pressure locally affects blood vessel compliance, which in turn affects the morphology of the pulse wave as measured by the sensing assembly. The morphology of the pulse waves will also depend at least partially on the user's blood pressure (e.g., the diastolic blood pressure and systolic blood pressure), and thus the relationship between applied pressure and pulse wave morphology (e.g., as measured using one or more metrics derived therefrom) may be indicative of a user's diastolic and systolic blood pressure (BP). Similarly, the relationship between the applied pressure and pulse wave morphology may be indicative a user's mean arterial pressure.

The morphology of each pulse wave may be analyzed in any suitable manner to generate the one or more metric values. In one example, a metric value may include an area under the curve (AUC) value that is determined for a respective pulse wave. Accordingly, each data point of the set of data points may include an AUC value calculated from a respective pulse wave. In these variations, the relationship between applied pressure and AUC values may be analyzed when determining a mean arterial pressure, a diastolic pressure and/or a systolic pressure associated with the measurement.

Additionally or alternatively, a metric value may include a pulse arrival time associated with a respective pulse wave. For example, an electronic device that incorporates the sensing assembly may be further configured to acquire an electrocardiograph (ECG) waveform during the measurement period. In these instances, a pulse arrival time value may be determined as a difference between i) a first time point at which the ECG waveform includes a predetermined feature associated with a heartbeat and ii) a second time point at which a pulse wave that corresponds to that heartbeat exhibits a predetermined morphological point. In these variations, each data point of the set of data points may include a pulse transit time value calculated from a respective pulse wave, and the relationship between applied pressure and pulse transit time may be analyzed when determining a mean arterial pressure, a diastolic pressure and/or a systolic pressure associated with the measurement.

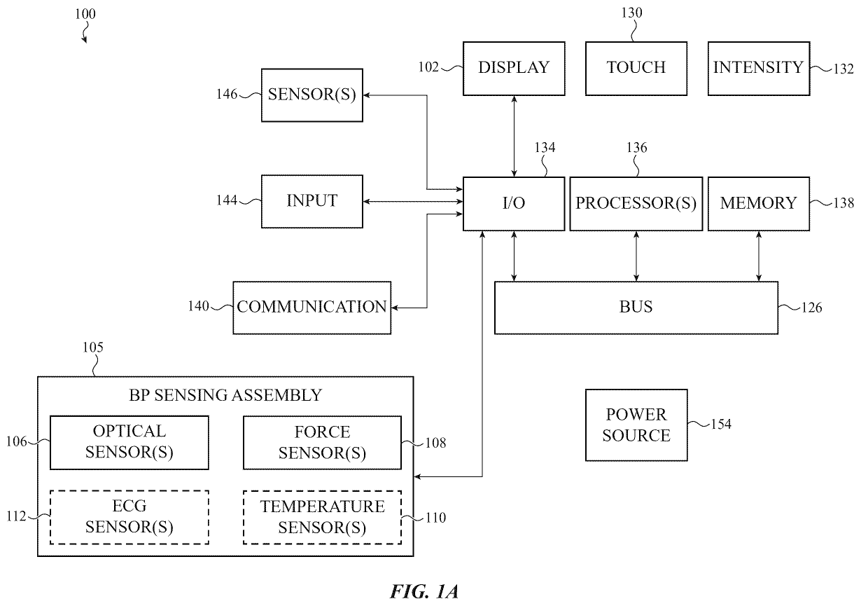

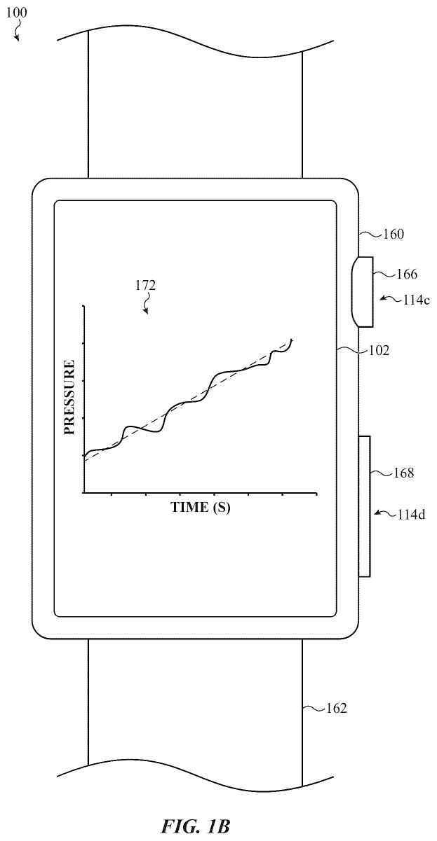

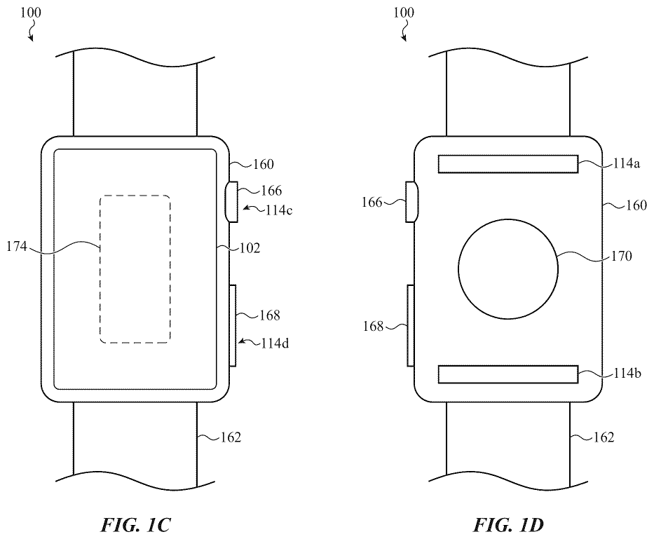

These and other embodiments are discussed below with reference to FIGS. 1A-10C. However, those skilled in the art will readily appreciate that the detailed description given herein with respect to these Figures is for explanatory purposes only and should not be construed as limiting.

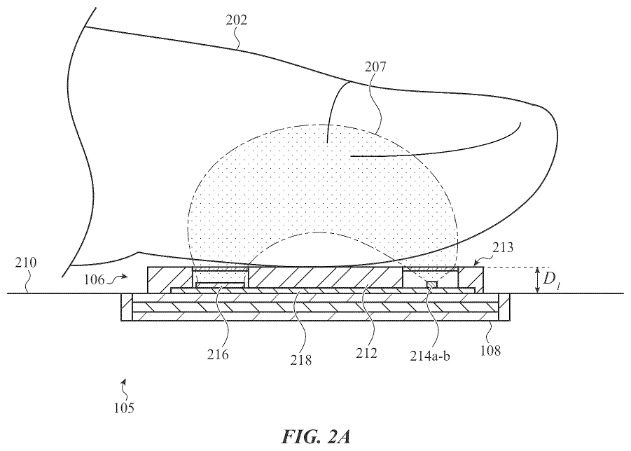

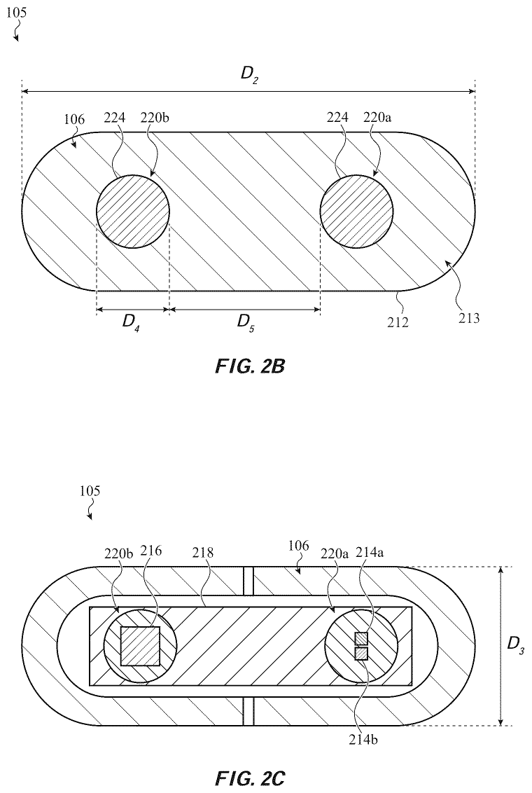

FIG. 1A depicts exemplary components of a device 100 that may be used to determine the blood pressure of a user according to the measurement techniques described herein. The device 100 includes a BP sensing assembly 105 that is configured to perform the BP measurement techniques described herein. Specifically, the BP sensing assembly 105 includes a set of one or more sensors that collectively perform BP measurements. For example, the BP sensing assembly 105 may include a set of one or more optical sensors 106, such as one or more photoplethysmography (PPG) sensors, laser doppler sensors, and/or other types of optical sensor(s). In some embodiments, one or more of the optical sensors 106 may include an optical sensor capable of performing both a PPG measurement and a laser doppler measurement. Each of the optical sensors 106 may generate a corresponding cardiac signal that represents a parameter associated with the volume or flow of blood in one or more blood vessels at each moment in time. As used herein, the term “cardiac signal” may refer to a signal associated with the volume of blood, changes in the volume of blood, blood flow, changes in blood flow, and/or other parameters or metrics associated with variations of blood in a portion of a user's body being measured by an optical sensor (e.g., the blood volume of the finger). Accordingly, in some instances a cardiac signal generated by the optical sensor(s) 106 may represent a direct measurement of blood volume (e.g., each value of the blood signal represents a volume of blood measured from the user) or may represent a relative change in blood volume (e.g., each value of the blood volume signal represents a change in volume relative to some baseline value). In some instances, the optical sensor(s) may be configured to measure multiple cardiac signals (e.g., a first cardiac signal the measures blood volume and a second cardiac signal that measures blood flow), each of which may be analyzed using the techniques described herein. Examples of the optical sensor(s) 106 are described in more detail herein, such as with respect to FIGS. 2A-C, 3A, 8A and 8B. While various embodiments are described herein with respect to generating measurement signals from the finger of a user (such as finger blood volume, variable pressure, etc.), it should be appreciated that the techniques described herein may be applied to other regions of a user's body (e.g., a skin region that includes a portion of a user's wrist, forehead, temple, or the like).

The BP sensing assembly 105 may include a set of one or more pressure sensors 108 for measuring force applied to the sensing assembly 105. The pressure sensor(s) 108 may generate a pressure signal that represents the pressure applied to the pressure sensor(s) 108 at each moment in time. In some examples the pressure signal may include, or may be, a force signal. For instance, the pressure sensor(s) 108 may be capable of converting measured pressure to force, or the pressure sensor(s) 108 may otherwise provide a force signal. In further examples, the pressure sensor(s) 108 may generate a force signal that may be converted to a pressure signal, such as by processor 136 and/or other components of device 100. In still other examples the pressure sensor(s) 108 may be a type of force sensor, and the force sensor may generate a force signal and/or a pressure signal.

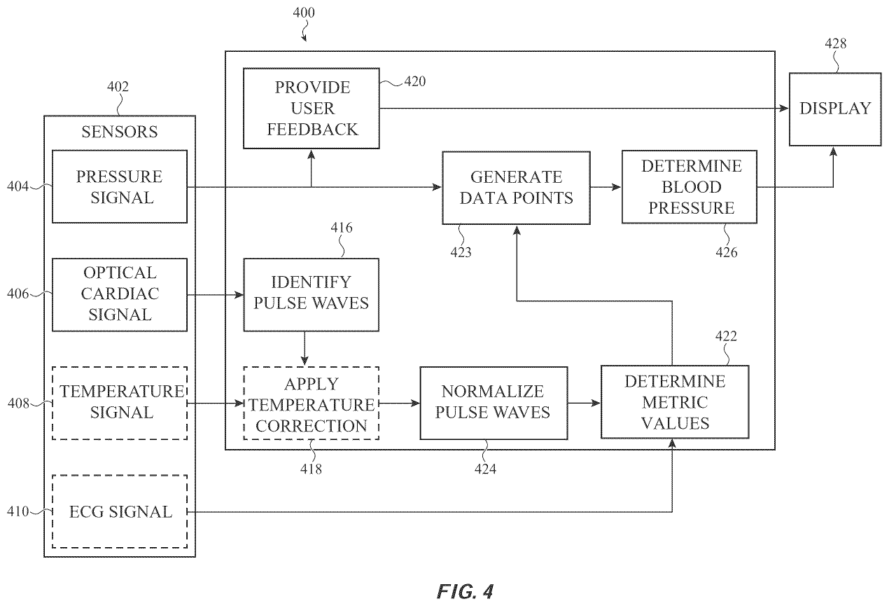

The BP sensing assembly 105 may optionally include a set of one or more temperature sensors 110 positioned to measure or estimate the temperature of the portion of the skin that is applying pressure to the sensing assembly 105, and that is being measured by the optical sensor(s) 106. The temperature sensor(s) 110 may include any of a wide variety of temperature-sensing components, such as a thermistor, thermocouple, thermopile, resistance temperature detector, and/or other types of temperature-sensing components. The temperature sensor(s) 110 may further include electronic components (e.g., analog and/or digital circuitry) for acquiring and processing temperature data from the temperature-sensing components. The temperature data may be used to compensate or correct data used to generate a BP estimate, which may be affected by finger temperature, as described below with respect to FIG. 4.

The BP sensing assembly 105 may optionally include an ECG sensor 112, which may be part of an ECG sensor system (such as may be included as part of sensors 146). For example, the ECG sensor 112 may include a plurality of electrodes arranged on one or more external surfaces the device 100, such as on a surface of the housing of the device 100 and/or input surfaces of the device 100 (e.g., the surface of a button). An example electrode arrangement is depicted in FIGS. 1B-1C and described below. In some cases, one or more electrodes may be positioned on a common surface through which the optical sensor(s) 106 measures blood volume signal. The ECG sensor(s) 112 may further include electronic components (e.g., analog and/or digital circuitry) connected to the electrode(s) for acquiring and processing signals associated with ECG.

The BP sensing assembly 105 may be included in portions of device 100 that provide access for the finger of the user. For example, the BP sensing assembly 105 may be part of a button, dial, crown, and/or other portion of a device 100. In some examples, the BP sensing assembly 105 may be part of an input mechanism 144, as described herein. Examples of a BP sensing assembly 105 are described with reference to FIGS. 2-4.

In some embodiments, device 100 has a bus 126 that operatively couples an I/O section 134 with one or more computer processors 136 and memory 138. I/O section 134 can be connected to display 102, which can have touch-sensitive component 130 and, optionally, intensity sensor 132 (e.g., contact intensity sensor). The touch-sensitive component 130 may be used to detect touch inputs applied to the surface of the display 102, including detecting locations of touch inputs, motions of touch inputs (e.g., the speed, direction, or other parameters a gesture applied to the cover can generate), or the like. Using the intensity sensor 132 the device 100 may detect amounts or magnitudes of pressure or force associated with touch events applied to the surface of the display 102. The touch-sensitive component 130 and/or intensity sensor 132 may, individually or in combination, detect various types of user inputs to control or modify the operation of the device 100, including taps, swipes, multiple finger inputs, single- or multiple-finger touch gestures, presses, and the like.

Further, the intensity sensor 132 may provide data associated with the force and/or pressure applied to the display 102 by the finger of a user. The touch-sensitive component 130 may also provide data associated with the contact surface area between the finger of the user and the surface of the display 102. In some instances, the intensity sensor 132 may act as the pressure (or force) sensor 108 of the sensing assembly 105 by providing a pressure signal or force signal that corresponds to the pressure or force applied to a portion of the display. The pressure sensor 108 of the sensing assembly 105 may be separate from the intensity sensor 132. The pressure or force signal provided by the intensity sensor 132 may be used as described herein as part of determining a user's BP.

The display 102 may define an output region in which graphical outputs are displayed. Graphical outputs may include graphical user interfaces, user interface elements (e.g., virtual buttons, sliders, etc.), text, lists, photographs, videos, or the like. In some cases, the display 102 may output a graphical user interface with one or more graphical objects that display information collected or derived from one or more sensors. For example, the display 102 may output information related to a measurement test, such as a type of test, a status of the test, measurement results, and so on.

In addition, I/O section 134 can be connected with communication unit 140 for sending and/or receiving application and operating system data, using Wi-Fi, Bluetooth, near field communication (NFC), cellular, and/or other wireless communication techniques.

Device 100 can include a set of one or more input mechanisms 144 operatively connected to the I/O section 134. In some examples, the input mechanism 144 may include a type of rotatable input device or a depressible and rotatable input device (such as the rotatable and depressible crown associated with a smartwatch). In other examples, the input mechanism 144 may include one or more buttons, dials, switches, knobs, levers, and/or other types of input mechanisms.

Device 100 includes various sensors 146, such as GPS sensor, accelerometer, directional sensor (e.g., compass), gyroscope, motion sensor, ambient temperature sensor, and/or a combination thereof, all of which can be operatively connected to I/O section 134. Some of these sensors, such as accelerometer 148 and gyroscope 152 may assist in determining an orientation of the device 100 or a portion thereof. Measurement signals acquired by sensors 146, or data associated with, or derived from, the acquired measurement signals, may be provided to the processor 136 via I/O section 134.

Memory 138 of device 100 can include one or more non-transitory computer-readable storage devices for storing computer-executable instructions, which, when executed by one or more computer processors 136, for example, can cause the processors 136 to perform the methods that are described herein. A computer-readable storage device can be any medium that can tangibly contain or store computer-executable instructions for use by or in connection with the instruction execution system, apparatus, or device. In some examples, the storage device is a transitory computer-readable storage medium. In some examples, the storage device is a non-transitory computer-readable storage medium. The non-transitory computer-readable storage device can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like.

The processor 136 can include, for example, a processor, a microprocessor, a graphics processing unit (GPU), a programmable logic array (PLA), a programmable array logic (PAL), a generic array logic (GAL), a complex programmable logic device (CPLD), an application-specific integrated circuit (ASIC), a field-programmable gate array (FPGA), or any other programmable logic device (PLD) configurable to execute an operating system and applications of device 100, as well as to facilitate blood pressure measurements as described herein. Device 100 is not limited to the components and configuration of FIG. 1, but can include other or additional components in multiple configurations.

Accordingly, any of the processes described herein may be stored as instructions on a non-transitory computer-readable storage device, such that a processor may utilize these instructions to perform the various steps of the processes described herein. Similarly, the devices described herein include a memory (e.g., memory 138) and one or more processors (e.g., processor 136) operatively coupled to the memory. The one or more processors may receive instructions from the memory and are configured to execute these instructions to perform the various steps of the processes described herein.

The device 100 further includes a power source 154, which may include electronic components that receive electrical power from an external electrical power system (e.g., from electrical mains), condition the electrical power, and/or distribute the electrical power to components of device 100. In examples where device 100 is a portable device, the power source 154 may include a type of battery or other portable source or electrical power, and components associated with charging the battery.

The power source 154 may include battery charging components, which may receive power, charge a battery of the device 100, and/or provide direct power to operate the device 100 regardless of the battery's state of charge (e.g., bypassing the battery). In some cases, the battery charging components may include a coil such that the device 100 may receive power wirelessly (e.g., via inductive power transfer). The device 100 may include a magnet, such as a permanent magnet, that magnetically couples to a magnet (e.g., a permanent magnet, electromagnet) or magnetic material (e.g., a ferromagnetic material such as iron, steel, or the like) in a charging dock (e.g., to facilitate wireless charging of the device 100) or other accessory device described herein.

FIGS. 1B and 1C depict front views and FIG. 1D depicts a rear view, respectively, of a variation of the device 100 of FIG. 1A, in which the device 100 is configured as a smartwatch. A smartwatch is merely one example of an electronic device that may be configured to perform the blood pressure measurements described herein, and the concepts discussed herein may apply equally or by analogy to other electronic devices, including, smart band, mobile phones (e.g., smartphones), tablet computers, notebook computers, head-mounted displays, headphones, earbuds, digital media players (e.g., mp3 players), or the like.

The device 100 includes a housing 160 and a band 162 coupled to the housing 160. The housing 160 may at least partially define an internal volume in which components of the device 100 may be positioned. The housing 160 may also define one or more exterior surfaces of the device, such as all or a portion of one or more side surfaces, a rear surface, a front surface, and the like. The housing 160 may be formed of any suitable material, such as metal (e.g., aluminum, steel, titanium, or the like), ceramic, polymer, glass, or the like. The band 162 may attach the device 100 to a user, such as to the user's arm or wrist.

The device 100 may also include one or more input mechanisms, such as described above for input mechanisms 144. For example, the device 100 may include a first input device 166 having a cap, crown, protruding portion, or component(s) or feature(s) positioned along a side surface of the housing 160. At least a portion of the first input device 166 (such as a crown body) may protrude from, or otherwise be located outside, the housing 160. In the variation shown in FIGS. 1B-1D, the first input device 166 is configured as a crown and may define a generally circular shape or circular exterior surface. The exterior surface of the first input device 166 may be textured, knurled, grooved, or otherwise have features that may improve the tactile feel of the first input device 166 and/or facilitate rotation sensing.

The first input device 166 may facilitate a variety of potential interactions. For example, the first input device 166 may be rotated by a user (e.g., the crown may receive rotational inputs). Rotational inputs of the first input device 166 may zoom, scroll, rotate, or otherwise manipulate a user interface or other object displayed on the display 102 among other possible functions. The first input device 166 may also be translated or pressed (e.g., axially) by the user. Translational or axial inputs may select highlighted objects or icons, cause a user interface to return to a previous menu or display, or activate or deactivate functions among other possible functions.

In some cases, the device 100 may sense touch inputs or gestures applied to the first input device 166, such as a finger sliding along the body of the first input device 166 (which may occur when first input device 166 is configured to not rotate) or a finger touching the body of the first input device 166. In such cases, sliding gestures may cause operations similar to the rotational inputs, and touches on a cap or crown may cause operations similar to the translational inputs. As used herein, rotational inputs include both rotational movements of the first input device 166, as well as sliding inputs that are produced when a user slides a finger or object along the surface of a crown in a manner that resembles a rotation (e.g., where the crown is fixed and/or does not freely rotate).

The device 100 may also include a second input device 168, which, in the example of FIGS. 1B-1D, is configured as a button. The second input device 168 may be a movable button or a touch-sensitive region of the housing 160. The button may control various aspects of the device 100. For example, the button may be used to select icons, items, or other objects displayed on the display 102, to activate or deactivate functions (e.g., to silence an alarm or alert), or the like.

In some examples, the first input device 166 and/or second input device 168 may include a BP sensing assembly 105. For instance, the first input device 166 may include the optical sensor(s) 106 and pressure sensor(s) 108, and thus may be capable of providing input signals used to estimate BP. The first input device 166 and/or second input device 168 may optionally include one or more temperature sensor(s), that may be used to account for the effects of finger temperature on the BP estimation.

In variations where the BP sensing assembly 105 of the device 100 includes an ECG sensor 112, the device 100 may include a set of electrodes 114a-114d (e.g., a first electrode 114a, a second electrode 114b, a third electrode 114c, and a fourth electrode 114d) for acquiring an ECG signal from a user. In other examples, the smartwatch (or other device) may include more or fewer electrodes. The electrodes of the set of electrodes 114a-114d may be positioned on any portions of the device 100 as may be needed to perform measurements using the sensors described herein.

For example, a rear-facing portion of the housing 212 (e.g., a surface of the housing 212 opposite the display 206) may include one or more electrodes. In the example depicted in FIG. 1D, a rear-facing surface of the device 100 includes two electrodes (e.g., the first and second electrodes 114a-114b) of the set of electrodes. In some examples, electrodes 114a-114b may be used to make contact with the user's wrist, other portion of the user's arm, or other portion of the user's body. In some examples, the rear-facing surface of the device 100 may include more or fewer electrodes than depicted in FIG. 1D. Further, the electrodes 114a-114b may be located on other portions of the housing 212, may be include electrodes with different shapes than depicted, and/or may be positioned and/or oriented on the housing 212 according to another arrangement.

In the example depicted in FIG. 1B-IC, the device 100 may include additional electrodes (e.g., the third and fourth electrodes 114c-114d) of the set of electrodes, such as for making contact with the finger of the user or with another portion of the user's body. For example, a surface of first input device 166 and/or second input device 168 may include electrodes 114c-114d, respectively. For example, the first input device 166 may include the third electrode 114c. In these instances, the third electrode 114c may be used in acquiring an ECG signal while the user contacts with the first input device 166. Additionally or alternatively, the second input device 168 may include the fourth electrode 114d, and the fourth electrode 114d may be used in acquiring an ECG signal while the user contacts the second input device 168.

In some variations, the surface of the first input device 166 and/or second input device 168 may include a single electrode (such as depicted), two electrodes, three electrodes, or more. In examples where a BP sensing assembly 105 is included as part of the first input device 166 and/or second input device 168, the electrodes 114c-114d may be included as part of each BP sensing assembly 105 (e.g., ECG sensor(s) 112). An example of a BP sensing assembly 105 included as part of the second input device 168 is described below with respect to FIGS. 2A-2C.

The electrodes 114a-114d may be conductively coupled to processing and/or other circuitry within the housing 160, such as described herein (e.g., associated with processor 136, sensor(s) 146, and/or the electrodes 114a-114d themselves). The electrodes 114a-d may be of any type of material suitable for forming electrodes. For instance, the electrodes 114a-d may be metal, ceramic, glass, plastic, composite, and/or other types of material. The electrodes 114a-d may be of the same type of material, or a subset of one or more of the electrodes 114a-d may be of different materials.

FIG. 1D shows a rear side of the device 100. The device 100 may include one or more windows 170 (one of which is shown) that allow light to pass through a portion of the housing 160. The one or more windows 170 may be part of an optical sensing system and coupled to the housing 160. The one or more windows 170 may include light transmissive materials and be associated with internal sensor components, which may be used to determine biometric information of a user, such as heart rate, blood oxygen concentrations, and the like, as well as information such as a distance from the smartwatch to an object. The particular arrangement of the one or more windows 170 in the housing 160 shown in FIG. 1D is one example arrangement, and other window arrangements (including different numbers, sizes, shapes, and/or positions of the windows) are also contemplated. As described herein, the window arrangement may be defined by or otherwise correspond to the arrangement of components in the integrated sensor package.

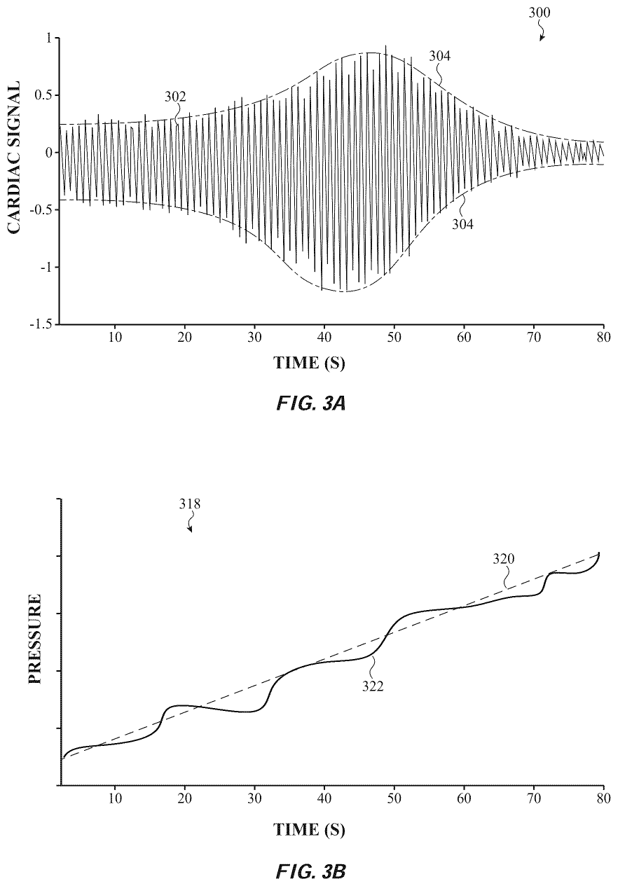

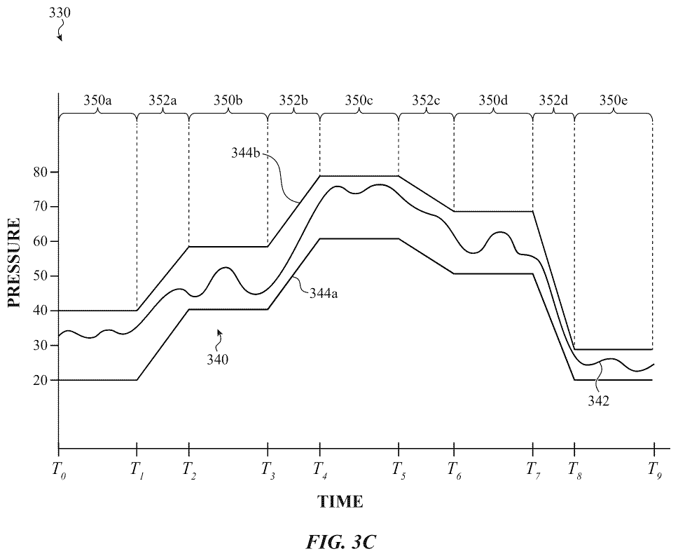

The device 100 includes a display 102, such as described above. The display 102 can be positioned at least partially within the housing 160 and used to provide graphical/visual output to the user (e.g., graphics, user interface elements, text, etc.), or to receive input from the user (e.g., touch input and/or pressure input). The display 102 may provide information about, and/or guide a user through, performing a blood pressure measurement. For instance, the display 102 may present a pressure input guide 172, which may provide visual guidance to the user for applying a variable pressure input during the measurement period of the blood pressure measurement. An example pressure input guide 172 is described in more detail below with respect to FIG. 3B.

As described herein, in some variations, the sensing assembly 105 may be included as part of the display 102. Accordingly, the display 102 may include an intensity sensor 132 capable of generating a pressure signal (or force signal) based on a pressure applied to the surface of the display 102. The device 100 may provide a visual indicator of a finger interface region 174 where the pressure may be applied. During a BP measurement session, the user may apply variable pressure within the finger interface region 174, and the intensity sensor 132 may provide a pressure signal corresponding to the applied pressure. The display 102 or other components of the device 100 may also determine the surface contact area between the finger of the user and the display 102 (e.g., using touch-sensitive components of the display, one or more imaging sensors, etc.). The determined contact area may be used, in some examples, to determine the pressure applied to the surface of the display 102.

In addition, when the device 100 is configured to perform blood pressure measurements via the display 102, the device 100 may further include an optical sensor or sensors (e.g., optical sensor(s) 106) positioned beneath the surface of the display 102, as described herein. The optical sensor(s) 106 provides a simultaneous blood volume measurement (or otherwise provides a cardiac signal) over the measurement period of the BP measurement session, such as when pressure is applied to the display 102.

In examples where components of the device 100 are used to collect pressure and blood volume (or other) measurements via the display 102, the presence of a user's skin against the display 102 may make it difficult for the user to view some or all of the display. Accordingly, the device 100 may provide non-visual feedback to the user to guide the application of variable pressure (such as a variable finger pressure). For example, the device 100 may provide haptic feedback, audio feedback, and/or other type of feedback that may assist the user in varying the amount of pressure applied by the user's finger. In other examples, the device 100 may provide data to another device (e.g., a smartphone, a computer, an external monitor, etc.) that may be in communication with the device 100. The other device may display a pressure input guide 172 for guiding the pressure applied by the finger of the user.

FIG. 2A depicts a cross-sectional side view of a variation of the BP sensing assembly 105 described herein with respect to FIGS. 1A-1D. As shown, the BP sensing assembly 105 may be incorporated into a device surface 210 that at least partially surrounds the BP sensing assembly 105. For example, the device surface 210 may be a portion of the device housing 160, such that the BP sensing assembly 105 may perform blood pressure measurements by pressing a skin region against the device housing 160. In the example depicted, the BP sensing assembly 105 is provided in the form of a button, such as second input device 168 of device 100. In these instances, the device surface 210 may represent a surface of the button that at least partially surrounds the BP sensing assembly. Accordingly, the BP sensing assembly 105 may perform blood pressure measurements by pressing a skin region against the button. For example, a finger 202 of a user is shown in FIG. 2A as positioned against the sensing assembly 105 as part of a blood pressure measurement.

In other examples, the sensing assembly 105 may be provided as part of another input mechanism (e.g., input mechanism 144) of the device, such as the first input device 166 (e.g., a crown body) or other input mechanism. In still other examples, the sensing assembly 105 may be part of a display of the device (e.g., display 102), such as described herein with reference to FIGS. 1A-1C. It should also be appreciated that a device as described herein may include multiple BP sensing assemblies, each incorporated into a different corresponding portion of the device.

The BP sensing assembly 105 includes at least an optical sensor 106 and a pressure sensor 108. The optical sensor 106 includes a sensor housing 212 that may at least partially define an internal volume in which components of the optical sensor 106 may be positioned. The sensor housing 212 includes an interface surface 213, upon which the user may apply pressure. The pressure sensor 108 may be configured to measure the pressure applied to the interface surface 213 during a blood pressure measurement.

The optical sensor is configured to provide illumination 207 to a skin region that is positioned against the interface surface 213 (e.g., anatomical structures within an interior volume of the finger 202) during a blood pressure measurement. The optical sensor 106 includes a set of optical emitters 214a-214b, such as a first optical emitter 214a and a second optical emitter 214b, that are each operable to emit light and thereby provide the illumination 207. Visualization of an example of the illumination 207 for both optical emitters 214a-214b is provided in FIG. 2A. The optical emitters 214a-214b may include any of a variety of light sources. For example, the optical emitters 214a-214b may include one or more light emitting diodes (LEDs), laser diodes, lamps, bulbs, and/or other types of light sources. While the example optical sensor 106 depicted in FIGS. 2A-C includes a plurality of optical emitters 214a-214b, in some examples, the optical sensor 106 may include a single optical emitter (e.g., a single LED or other type of light source). In further examples, the optical sensor 106 may include three or more optical emitters. The optical emitters 214a-214b may be capable of providing illumination 207 over one or more wavelengths. For instance, one or more of the optical emitters 214a-214b may provide illumination 207 in the visible spectrum (e.g., green, red, yellow, etc.), infrared (IR) spectrum, and/or other spectrum of light.

The optical sensor 106 includes at least one photodetector 216 positioned to receive and measure light emitted by the optical emitters 214a-214b after the light has interacted with the skin and other tissues of the finger 202 and may thereby generate one or more cardiac signals. In examples where the optical sensor 106 includes a type of PPG sensor, the intensity of light received by the photodetector 216 is proportional to changes in blood volume. In these instances, the optical sensor 106 may generate a cardiac signal that represents the changes in blood volume over time. Additionally or alternatively, such as in examples where the optical sensor 106 includes a laser doppler-based sensor, the light received by the photodetector 216 may be associated with blood flow through the vasculature of the finger 202. In these instances, the optical sensor 106 may generate a cardiac signal that represents a measured blood flow. In some variations, the photodetector 216 may be used to generate multiple cardiac signals that includes information associated with both blood volume (e.g., from PPG measurements) and blood flow (e.g., from laser doppler measurements). In such variations, one or more optical emitters may be configured to provide illumination 207 suitable for performing both type of measurements.

The photodetector 216 may be comprised of one or more photodiodes, phototransistors, complimentary metal-oxide-semiconductor (CMOS) sensors, and/or other types of photodetectors.

The optical emitters 214a-214b and photodetector 216 may be mounted to a printed circuit board (PCB) 218, which may be a type of rigid, rigid-flex, flex, or other type of PCB. In some examples, the optical emitters 214a-214b may be mounted on a separate PCB than the photodetector 216, and/or different emitters of the optical emitters 214a-214b may be mounted on separate PCBs from each other. The PCB 218 may include passive and/or active electronic components associated with the transmission of light from the optical emitters 214a-214b and/or the processing of signals generated by the photodetector 216 in response to the received light.

FIGS. 2B-2C depict additional details of the optical sensor 106 and components thereof. FIG. 2B depicts a top view of the BP sensing assembly 105 (including the optical sensor 106), that includes the sensor housing 212. FIG. 2C depicts a top view of the BP sensing assembly 105 with sensor housing 212 removed, such that components of the optical sensor 106 are visible.

FIG. 2C depicts an example arrangement in which optical emitters 214a-214b are located together within a first aperture 220a of the sensor housing 212, and the photodetector 216 is located within a second aperture 220b of the sensor housing 212. However, a wide variety of arrangements and/or combinations of the optical emitters 214a-214b and photodetector 216 are possible within each aperture 220a-220b. As described above, the optical sensor 106 may include a greater or fewer number of optical emitters and/or a greater number of photodetectors. In some examples, each aperture 220a-220b may include one or more photodetectors and/or one or more optical emitters. For instance, the optical sensor 106 may include one or more arrays of photodetectors and/or one or more arrays of optical emitters. The optical emitters 214a-214b and photodetector 216 may be arranged, grouped, and/or combined within the apertures 220a-220b, and the number of optical emitters 214a-214b and photodetector(s) 216 selected, to achieve a desired illumination 207 of the finger 202.

As depicted in FIG. 2B, the apertures 220a-220b may have the same diameter D4, or may, in other examples, may have different diameters. The diameter D4 of the apertures 220a-220b may be selected based on optical performance of the optical emitters 214a-214b and/or photodetector 216, or may be based on other factors.

The apertures 220a-220b may be separated by a distance D5. As described herein, the distance D5 may be selected to achieve a desired illumination 207 of the finger 202. For example, the distance D5 may be chosen to achieve a desired illumination intensity at the photodetector 216.

In addition, the optical sensor 106 may include a greater number of apertures than depicted in FIGS. 2A-2C, and the apertures may be arranged differently than shown (e.g., may not be arranged in a line). For example, the optical sensor 106 may include a total of three or more apertures, and the apertures may be positioned in a triangular, circular, square, or other arrangement. In further examples, the optical sensor 106 may include a single aperture through which the optical emitters 214a-214b transmit light and the photodetector 216 receives light. The number of apertures, the distance between the apertures (e.g., distance D5), the diameter of the apertures (e.g., diameter D4), and/or other parameters of the apertures and/or optical components may be selected to achieve a desired optical performance. For instance, these parameters may be selected to establish one or more optical pathways that may improve the accuracy of the cardiac signal. For example, configuring the optical sensor 106 to include multiple different optical pathways (e.g., by including multiple apertures that through which different photodetectors receive light, by including an array of photodetectors associated with a given aperture, or the like) may reduce the sensitivity of the cardiac signal to anatomic variations of the tissue being measured (e.g., size of a fingertip being measured, location and size of arteries of the fingertip, etc.).

The apertures 220a-220b may include an optical window material 224 that protects the optical emitters 214a-214b and photodetector 216 from the external environment, while allowing for the passage of light. The window material 224 may be an optically transparent or translucent material, such as a type of plastic, glass, ceramic, crystal, composite, or other type of material. The window material 224 may include features and/or properties that affect the passage of light through the window material 224. As an example, the window material 224 may be, or may include one or more filters, lens, and/or other optical components that improve the accuracy of the cardiac signal.

In addition, the sensor housing 212 may be designed to provide a suitable surface area for receiving the force applied by the finger 202. For example, the length D2 and width D3 of the sensor housing 212, along with shape of the interface surface 213 (e.g., a pill-shaped interface surface 213 is shown in FIGS. 2B and 2C), may be designed to achieve a suitable surface area for measuring applied force and/or pressure. The surface area of the housing 212 may be used to determine the pressure applied by the finger 202, such as when the pressure sensor 108 is a type of force sensor.

Returning to FIG. 2A, to reduce the possibility of physical interaction between the finger 202 and the device surface 210 that may unintentionally impact the force applied to the BP sensing assembly 105, the BP sensing assembly 105 may be positioned such that the interface surface 213 of the sensor housing 212 is positioned a distance D1 above the surrounding device surface 210. In further examples the distance D1 may be more than 3 mm. In examples where the interface surface 213 is substantially flush with the device surface 210, the finger 202 may come into contact with the device surface 210, which may affect the pressure transmitted to the pressure sensor 108.

The pressure sensor 108 may be any of a variety of sensors suitable for measuring pressure and/or force applied to the interface surface 213. For example, the pressure sensor 108 may be a type of strain gauge, load cell, micro-electromechanical system (MEMS), manometer, piezoelectric, capacitive, resistive, inductive, or other type of pressure/force sensor. In examples where the pressure sensor 108 outputs a signal corresponding to the applied force, the pressure sensor 108 (or other component of the device 100) may use the surface area of the sensor housing 212 to determine a pressure. The pressure sensor 108, PCB 218, and/or other components or systems of the device 100 may include passive and/or active circuit components associated with processing the measured pressure (or force) to generate an output pressure (or force) signal associated with the pressure applied by the finger 202. An example pressure signal is depicted in FIG. 3B and described in more detail below.

In examples where the device 100 includes an ECG feature, the BP sensing assembly 105 may include features or components that support measurement of an ECG signal. For example, the sensor housing 212 may include a set of one or more conductive regions, or may be entirely conductive, such that the sensor housing 212 may function as a set of one or more electrodes. Conductive regions of the sensor housing 212 may be electrically connected to circuitry (not depicted) associated with the ECG feature.

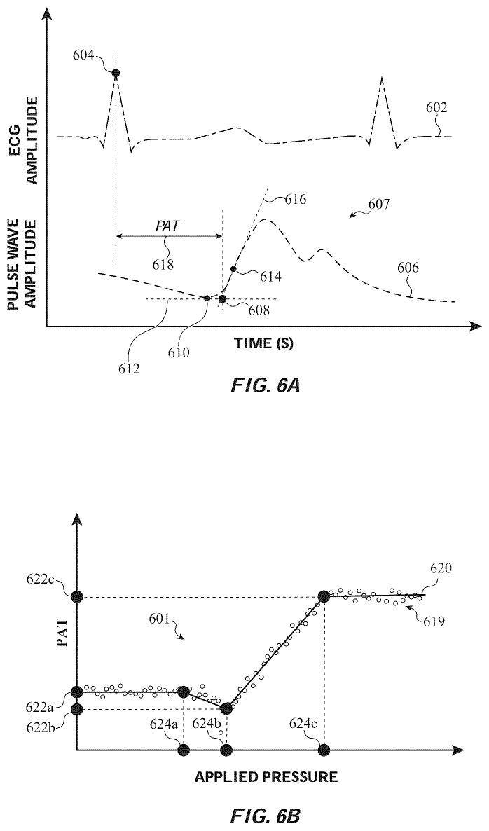

As described herein, the blood pressure measurements performed by the device (e.g., device 100) utilize a cardiac signal and a pressure (or force) signal that are obtained over a common measurement period. FIG. 3A depicts a graph 300 that includes an example cardiac signal 302, such as may be acquired from the finger of a user over a measurement period by an optical sensor (e.g., optical sensor 106) associated with a BP sensing assembly (e.g., BP sensing assembly 105) of a device. In the example depicted, the cardiac signal 302 may be expressed as a change in blood volume. In other examples, the cardiac signal may be expressed as a measured blood volume, a blood flow, or the like, as described herein. While the cardiac signal 302 is depicted in FIG. 3A as being measured over an 80 second period, in other examples the measurement period may be longer or shorter. The oscillations of the cardiac signal 302 represent the change in blood volume that results from pressure wave generated by cardiac contraction. These oscillations are referred to herein as pulse waves. Example pulse waves are depicted in greater detail in FIGS. 6A-6B. The oscillation amplitude of the pulse waves within the cardiac signal 302 can be represented by an envelope 304, which approximates how the amplitude changes over the course of the measurement period.

As described herein, the cardiac signal 302 is acquired from a finger of a user while a varying pressure is applied by the finger. FIG. 3B depicts a graph 318 that includes an example pressure signal 322, such as may be acquired over the measurement period by a pressure sensor (e.g., pressure sensor 108). In some examples, the device 100 may provide graph 318 on a display (e.g., display 102) during the measurement period, as a method of providing guidance or feedback to the user as the varying pressure is applied. In further examples, the graph 318 may include a pressure profile 320 that serves as a visual reference to the user of a target amount or range of pressures for the user to apply over the course of the measurement period. The graph 318 may display the current value of the pressure signal 322 in real-time or near real time, in relation to the pressure profile 320, as the pressure is being applied.

The pressure profile 320 may include a target pressure, which may be represented as a single target value or by a range of target values at a given moment in time. When the target pressure is represented by a single target value, a user may attempt to apply a pressure that meets the target value. When the target pressure is represented by a range of target values, a user may attempt to apply a pressure that falls within the range of target values. In some variations, the pressure profile 320 may be configured such that the target pressure changes across a measurement. In the example depicted in FIG. 3B, pressure is applied by the finger of the user according to a pressure profile 320 with a target pressure that is continuously linearly increasing. In other examples, the target pressure may be varied according to another pressure profile. For example, the pressure profile 320 may be one or more periods (or a portion of one period) of a sinusoid, a series of discrete steps (stepping up or down in pressure), a portion of a parabola (up or down), or any of a wide variety of suitable pressure profiles.

In other variations, the pressure profile 320 may include a target pressure that is constant over at least a portion of the measurement (e.g., the target value or range of target values does not change during this portion of the measurement). In these instances, the pressure applied by a user may naturally vary as the user attempts to apply the target pressure, which may allow the pressure sensor to measure a range of pressure values even as the target pressure remains constant. In some of these variations, if the natural variations in the pressure applied by the use do not cover a predetermined range of pressures during a certain period of time, the device may update the pressure profile (e.g., to a second target pressure value that is constant over a subsequent portion of the measurement, or to a varying target pressure).

In some instances, pressure guidance may be provided to the user by non-visual methods. For example, the device may provide an audible tone or tones that represent the amount of pressure applied by the user. In another example, the device may provide a type of haptic or other form of guidance to the user for applying variable pressure.

The pressure profile selected for a given measurement may be selected to obtain a plurality of pressure measurements that span a predetermined range of pressures. For example, it may be desirable to measure an overall range of pressure values that includes one or more values that are expected to be below a user's diastolic pressure and one or more values that are expected to be above a user's systolic pressure. Accordingly, the pressure profile may be selected that includes target pressures encompassing this range.

FIG. 3C shows a graph 330 of another variation of pressure profile 340 that may be used during a blood pressure measurement as described herein. Specifically, the pressure profile 340 may include, at each moment in time, a target pressure that includes a range of target values. As shown in FIG. 3C, the range of target values may be defined by a lower target value 344a and an upper target value 344b. During a blood pressure measurement, a user may attempt to apply a pressure (e.g., by a skin region pressed against the BP sensing assembly) that stays between the lower target value 344a and the upper target value 344b during the blood pressure measurement. In the variation shown in FIG. 3C, the pressure profile 340 is configured such that the lower target value 344a and the upper target value 344b change over the course of the measurement.

Specifically, the pressure profile 340 is configured as a series of steps, each of which has a corresponding constant target pressure, where the target pressure is defined by a corresponding range of target values. For the purpose of illustration, the graph 330 has been labeled with pressure values ranging between 0 and 100, which represent normalized pressure values in which 0 represents a minimum pressure value that may be measured by the BP sensing assembly and 100 represents a maximum pressure value that may be measured by the BP sensing assembly. In the variation shown in FIG. 3C, the pressure profile 340 is configured to obtain a plurality of pressure measurements that span an overall target range of pressure values between a lower pressure boundary (e.g., 20) and an upper pressure boundary (e.g., 80), and includes a plurality of steps 350a-350c. Each step of the pressure profile 340 includes a corresponding target pressure that encompasses a subset of the overall target range of pressure values.

In some variations, the plurality of steps 350a-350e includes a first group of steps 350a-350c that collectively encompass the overall target range of pressure values. For example, the first group may include a first step 350a (occurring between time T₀ and time T₁), a second step 350b (occurring between time T₂ and time T₃), and a third step 350c (occurring between time T₄ and time T₅) of the plurality of steps 350a-350e, though it should be appreciated that the first group may include more or fewer steps as may be desired. Specifically, the first step 350a may be associated with a first subset of the overall pressure range (e.g., a first range of target values in which the lower target value 344a is 20 and the upper target value 344b is 40). Similarly, the second step 350b may be associated with a second subset of the overall pressure range (e.g., a second range of target values in which the lower target value 344a is 40 and the upper target value 344b is 60) and the third step 350c may be associated with a third subset of the overall pressure range (e.g., a third range of target values in which the lower target value 344a is 60 and the upper target value 344b is 80).

In the variation shown in FIG. 3C, the ranges of target values corresponding to the first group of steps 350a-350c border without overlapping. Specifically, the upper target value of the first step 350a is the same as the lower target value of the second step 350b and the upper target value of the second step 350b is the same as the lower target value for the third step 350c. In other variations, however, two or more steps within the first group of steps 350a-350c may have corresponding ranges of target values that at least partially overlap. For example, the first range of target values corresponding to the first step 350a may at least partially overlap with the second range of target values corresponding to the second step 350b and/or the second range of target values corresponding to the second step 350b may at least partially overlap with the third range of target values corresponding to the third step 350c.

Additionally, the pressure profile 340 may be configured to move between the first group of steps 350a-350c in any order as may be desired. For example, the first group of steps 350a-350c may be configured such that the target pressure of the pressure profile 340 user progressively increases between successive steps such as shown in FIG. 3C (e.g., increases between the first step 350a and second step 350b and further increases between the second step 350b and the third step 350c). In other variations, the first group of steps 350a-350c may be configured such that the target pressure progressively decreases between successive steps (e.g., decreases between the first step 350a and second step 350b and further decreases between the second step 350b and the third step 350c). In still other variations, the first group of steps 350a-350c may be ordered such that the target pressure increases between some successive steps (e.g., between the first step 350a and the second step 350b) and the target pressure decreases between other successive steps (e.g., between the second step 350b and the third step 350c).

The pressure profile 340 may be configured to transition between successive steps of the plurality of steps 350a-350e in any suitable manner. For example, in the variation shown in FIG. 3C, the pressure profile 340 is configured to continuously change the target pressure between successive steps of the plurality of steps 350a-350e. Specifically, the pressure profile 340 may include a set of transition periods 352a-352d, each of which continuously transitions the target pressure between successive steps of the plurality of steps 350a-350e. For example, the set of transition periods 352a-352d includes a first transition period 352a (between time T₁ and time T₂) that links the first step 350a to the second step 350b. The first transition period 352a continuously transitions the target pressure from the first range of target values to the second range of target values (e.g., the lower target value 344a continuously increases from 20 to 40 and the upper target value 344b continuously increases from 40 to 60). Similarly, the second set of transition periods 352a-352d may include a second transition period 352b (between time T₃ and time T₄) linking the second step 350b to the third step 350c (e.g., such that the lower target value 344a increases from 40 to 60 and the upper target value 344b increases from 60 to 80). Transitioning the target pressure in this way may help to guide the user to increase or decrease the applied pressure toward the new target pressure. It should be appreciated that the BP sensing assembly may continue to perform measurements during these transitions, and thus may generate one or more data points corresponding to these transitions.

In some variations, the pressure profile 340 may be additionally or alternatively be configured to include a discontinuous target pressure jump between one or more pairs of successive steps of the plurality of steps 350a-350c. Using the first step 350a and the second step 350b as an example, the pressure profile 340 may be configured such that the second step 350b begins at the same time the first step 350a terminates (e.g., at time T₁). In these instances, the target pressure of pressure profile 340 will jump from the first target pressure to the second target pressure (e.g., the lower target value 344a jumps from 20 to 40 at time T₁ and the upper target value 344b jumps from 40 to 60 at T₁).

In some variations, the pressure profile 340 may be dynamically updated over the course of a measurement. For example, in some variations the duration of a given step of the plurality of steps 350a-350e may be adjusted depending on one or more measurements performed during that step. For example, it may be desirable to generate, for a given step of the pressure profile 340, a set of data points that meet certain measurement criteria (e.g., measurement of at least a minimum number of pulse waves, satisfy a particular distribution of pressure values associated with the measured pulse waves, or the like). Using the first step 350a as an example, the first step 350a may be configured to have a corresponding duration that is variable between a minimum duration and a maximum duration. In these instances, the first step 350a may continue for at least the minimum duration. If the measurement criteria are met prior to the first step 350a reaching the maximum duration, the first step 350a may terminate before the maximum duration. If, however, the measurement criteria are not met prior to the first step 350a reaching the maximum duration, the first step 350a may terminate at the maximum duration.

Additionally or alternatively, the pressure profile 340 may be dynamically updated based in least in part on the results of one or more completed steps of the plurality steps 350a-350c. For example, the pressure profile 340 may be configured to perform the first group of steps 350a-350c by default (e.g., the first groups of steps 350a-350c is performed each time the pressure profile 340 is selected for a given blood pressure measurement). Depending on the data points that are collected from the first group of steps 350a-350c (e.g., generated from measurements performed during these steps), the pressure profile 340 may be updated to include one or more additional steps. If the BP sensing assembly determines that the data points collected from the first group of steps 350a-350c (and, in some instances, data points collected from transition periods linking the first group of steps 350a-350c, such as the first and second transition periods 352a, 352b) are sufficient to complete the blood pressure measurement, the blood pressure measurement session may terminate after the first group of steps 350a-350c.

If however, the BP sensing assembly determines that additional data points are required to complete the blood pressure measurement, the pressure profile 340 may be updated to include one or more additional steps. For example, the actual pressure applied by the user (as indicated by pressure signal 342 measured by the BP sensing assembly) during a given step may have not sufficiently covered the range of target values and/or may have fallen outside of the range of target values for that step. In some instances, the pressure profile 340 may be updated such that the plurality of steps 350a-350e includes a second group of steps 350d-350c. In the example shown in FIG. 3C, the second group of steps 350d-350e may include a fourth step 350d (between time T₆ and time T₇) and a fifth step 350e (between time T₈ and time T₉). The target pressures and/or durations of these additional steps may be selected based on what data points are needed to complete the blood pressure measurement.

For example, if the BP sensing assembly determines that additional data points are needed at pressure values between 50 and 70, the fourth step 350d may be set with a target pressure having a fourth range of target values in which the lower target value 344a is 50 and the upper target value 344b is 70. Similarly, if the BP sensing assembly determines that additional data points are needed at pressure values between 20 and 30, the fourth step 350d may be set with a target pressure having a fifth range of target values in which the lower target value 344a is 20 and the upper target value 344b is 30.

When dynamically updating a pressure profile as described herein (e.g., pressure profile 340), it may be desirable to update the pressure profile in a way that reduces the impact of hysteresis in the cardiac signal generated by an optical sensor (e.g., optical sensor 106) of the BP sensing assembly. Aspects of the cardiac signal may be different, during a given step of the pressure profile 340 of FIG. 3C, depending on aspects of the pressure applied by a user before that step, which may impact the data points generated as part of a measurement session. For example, the vasodilation of tissue and/or the amount of tissue fluid present in a skin region at a given pressure level may depend at least in part on whether the skin region had previously experienced relatively higher or relatively lower pressure. Accordingly, when dynamically adding the second group of steps 350d-350e to the pressure profile 340, the order of these additional steps, the duration of each additional step, and/or the rates of transition between successive steps (e.g., a duration of a third transition period 352c between the third step 350c and the fourth step 350d and/or a duration of a fourth transition period 352d between the fourth step 350d and the fifth step 350c) may be selected to reduce the impact of cardiac signal hysteresis in the resulting blood pressure measurement.

It should be appreciated that different pressure profiles may be selected for different measurement sessions. For example, for a first measurement session, a first pressure profile may be selected that is configured to collect a first overall range of target pressures having a first upper pressure boundary and a first lower pressure boundary. The first pressure profile includes one or more target pressures (e.g., as part of one or more steps of the pressure profile 340 of FIG. 3C) that encompass each of the first upper pressure boundary and the first lower pressure boundary. For a second measurement session, a second pressure profile may be selected that is configured to collect a second overall range of target pressures different that the first overall range of target pressures. Specifically, the second overall range of target pressures may have a second upper pressure boundary and a second lower pressure boundary, where at least one of these limits is different than the corresponding limits of the first overall range of target pressures. The second pressure profile includes one or more target pressures that encompass each of the second upper pressure boundary and the second lower pressure boundary. In this way, the pressure profile for each measurement session may be tailored to the needs of that individual measurement session, such as described in more detail herein.

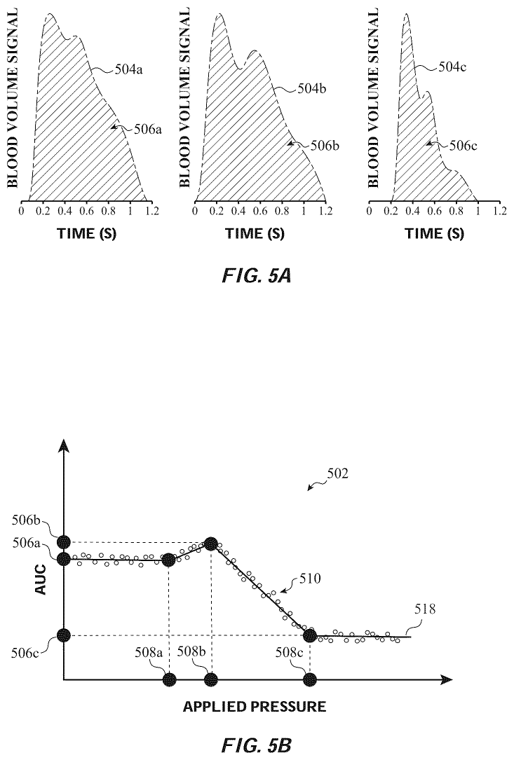

As described above, varying the applied pressure affects at least some of the properties and parameters of the vessels in the region of the finger where the pressure is applied. For example, compliance of the vessels in the finger may be a function of the amount of pressure applied. Thus, as depicted in FIG. 3A, the amplitude of the cardiac signal 302 (shown in FIG. 3A as a blood volume signal) may vary over a set of one or more ranges of applied pressure. Additionally or alternatively, other morphologic features of each pulse wave may be affected by the applied pressure. A set of one or more metrics corresponding to one or more of the morphologic pulse wave features may be used to determine the systolic and diastolic pressures of the user. Examples of morphologic changes in the pulse waves, and corresponding metrics, are depicted in FIGS. 5A-5B and 6A-6B and described below.

FIG. 4 depicts an example process 400 for determining systolic and diastolic blood pressures based on a set of one or more metrics derived from one or more morphologie features of the cardiac signal (e.g., cardiac signal 302). The example process 400 may be performed by one or more processors as described herein (e.g., processor 136). Process 400 may be performed as a method, or may be stored as instructions on a non-transitory computer-readable storage device, such that the processor may utilize these instructions to perform the various steps of the processes described herein. Further, the processor may be operatively coupled to a memory (e.g., memory 138), where the processor is configured to execute instructions that cause the processor to perform process 400.

As part of process 400, signals 404-410 may be received from a set of one or more sensors 402, which may include sensors associated with a BP sensing assembly (e.g., BP sensing assembly 105). As described herein, the sensors 402 may include at least one pressure/force sensor (e.g., pressure sensor 108) that may be operated to generate a pressure signal 404 from a skin region of the user, at least one optical sensor (e.g., optical sensor 106) that generates a cardiac signal 406 from the skin region of the user, an optional temperature sensor or sensors (e.g., temperature sensor 110) that generates a temperature signal 408, and an optional ECG sensor (e.g., ECG sensor 112) that generates an ECG signal 410. Each of the signals 404-410, obtained over a measurement period, may include a series of values, each of which corresponds to a different point in time within the measurement period. The signals 404-410 generated by sensors 402 may undergo one or more initial processing steps before they are utilized as part of process 400. The initial filtering may include filtering, denoising, and/or other types of initial processing.

As described herein, during the measurement period, the user applies pressure to the sensing assembly via the skin region of the user and a pressure (or force) sensor (e.g., pressure sensor 108) outputs pressure signal 404 accordingly, that represents the pressure applied by the skin region to the sensing assembly. A visual guide (e.g., pressure profile 320 or pressure profile 340) may be provided on the device display 428, so that the user may understand how to apply the pressure (e.g., linearly increasing, sinusoidal, etc.). At operation 420, process 400 may use pressure signal 404 to provide feedback to the user in real-time or near real-time. The user may interpret the feedback and attempt to apply the correct amount of pressure over the course of the measurement period. For example, process 400 may provide feedback to the display 428 in the form of a graphic that indicates the current and previous pressure applied by the user, such as relative to the pressure profile. As the measurement period continues, process 400 may update the graphic to indicate the current pressure being applied. Accordingly, the user may be guided to apply pressure across a range of values that are suitable for determining blood pressure, as described herein.

Additionally or alternatively, process 400 may provide a non-visual form of feedback to the user. For instance, process 400 may provide auditory, haptic, and/or other forms of feedback to the user, indicating whether the user is applying a suitable amount of pressure to the sensing assembly. In further instances, the feedback may help guide the user in how to apply the pressure. As an example, process 400 may provide a variable frequency audible tone that corresponds to the amount of pressure to apply to the sensing assembly (e.g., frequency increases may indicate to apply greater pressure, frequency decreases may indicate to apply less pressure). As another example, process 400 may provide a variable strength haptic stimulus that corresponds to the pressure to be applied to the sensing assembly. In other examples, process 400 may provide still other forms of feedback that indicate the applied pressure currently being applied and/or pressure to apply to the sensing assembly. At operation 416, the process 400 identifies pulse waves using the optical cardiac signal 406. Each of the pulse waves corresponds to a blood pressure wave transmitted through the vasculature as a result of a cardiac contraction (e.g., a heartbeat). The pulse waves may be identified using any of a variety of established methods for identifying a recurring waveshape or set of pulses within a signal. For example, process 400 may apply one or more amplitude thresholds and/or timing thresholds to identify the pulse waves based on amplitude and/or timing characteristics of each of the pulse waves. In other examples, the process 400 may use time-domain techniques, frequency-domain techniques, and/or other techniques or methods to identify the pulse waves. Examples of pulse waves are provided with respect to FIG. 5A.

In some instances, certain environmental factors may also impact the morphology of individual pulse waves. For example, the morphology of a given pulse wave may at least partially depend on the temperature of the tissue being measured. Accordingly, it may be desirable to account for these temperature-induced changes in the morphology of the pulse waves identified in operation 416. In some variations, the process 400 may, at operation 418, apply temperature correction to the identified pulse waves using the temperature signal 408 measured during the measurement period.

Temperature correction may be implemented as a mathematical function or other type of algorithm that uses temperature values from the temperature signal 408 to correct the amplitude and/or duration of the pulse waves. A temperature value may be determined from portions of the temperature signal 408 that correspond to a given pulse wave (e.g., the temperature value may correspond to a value from the temperature signal, or may be derived from multiple values of the temperature signal 408). The temperature values used to perform the temperature correction may be i) skin temperature values (e.g., from a temperature measurement of the skin in contact with the sensing assembly 105), ii) ambient temperature values measured from a temperature sensor of the device (e.g., an ambient temperature sensor associated with sensors 146), or iii) a combination of skin temperature, ambient temperature, and/or other measured temperature values provided as part of the temperature signal 408.

In some variations, analysis of the temperature signal 408 may include a determination that one or more conditions exist such that may impact the ability of the pulse waves to be effectively temperature compensated. For instance, analysis of the temperature signal 408 may indicate a skin temperature and/or ambient temperature that is too low for effective temperature compensation. In some of these instances, the process 400 may forego or otherwise terminate the blood pressure measurement, and may provide an alert and/or notification indicating a blood pressure measurement is not currently available.

Process 400 further includes, at step 424, normalizing the pulse waves or, if temperature correction is applied, normalizing the temperature-corrected pulse waves. Normalizing the pulse waves may allow the morphologic features of each of the series of pulse waves to be compared on a similar scale. In one example, the normalization may include scaling the amplitude of each of the pulse waves (or temperature-corrected pulse waves) relative to the amplitude of one of the pulse waves. For instance, the amplitude of the pulse waves may be scaled relative to the amplitude of the largest pulse wave. The scaling may result in normalized pulse waves that are scaled to a range between 0 and 1, where a value of 1 is equivalent to the amplitude of the largest amplitude pulse wave in the series of pulse waves. In other instances, the normalized pulse waves may be scaled to a different range.

In additional examples, the pulse waves may be normalized according to any of a variety of normalization methods. In examples, the normalization may include maximum absolute value normalization, root mean square (RMS) normalization, normalizing using a probability distribution, and/or other methods for performing normalization. The normalized pulse waves are used to determine metric values, as described below.

At operation 422, the process 400 uses the normalized pulse waves and (optionally) the ECG signal 410, to determine metric values associated with the morphology of each normalized pulse wave. As one example, determining a metric value may include determining the area under the curve (AUC) of each of the normalized pulse waves, such as by performing a type of mathematical integration.

Additionally or alternatively, determining a metric value (at operation 422) may include determining a pulse arrival time (PAT) using the normalized pulse waves and ECG signal 410 (if provided). For example, the PAT may be affected by the morphology of the normalized pulse waves and may be used as a metric to help determine the user's blood pressure. Determination of PAT is described below with respect to FIG. 6A. Briefly, the process 400 may identify a series of R-waves within the ECG signal 410, where each R-wave precedes a corresponding pulse wave. For each of the identified R-waves and corresponding normalized pulse waves, the PAT may be determined as the time between the R-wave and a point associated with the normalized pulse wave (e.g., the base or “foot” of each normalized pulse wave). The PAT may be used as a metric value for generating data points, as described herein with reference to operation 423.

At operation 423, the process 400 may use the determined metric values and the pressure signal 404 to generate a set of data points, where each data point includes a determined metric value and a pressure value taken from the pressure signal 404. In some cases, each data point may include a determined metric value and pressure value calculated or derived from the pressure signal 404. For example, the process 400 may associate the AUC of each of the normalized pulse waves with a pressure value from the pressure signal 404. For instance, a data point generated as part of process 400 may include a value for the AUC for a normalized pulse wave and a pressure value acquired during the occurrence of the corresponding normalized pulse wave. In some variations, the pressure value may be selected or derived from the pressure values acquired during the occurrence of the corresponding normalized pulse wave. In one example, the pressure value used to generate a data point may be the maximum pressure value that occurs during the corresponding normalized pulse wave. Accordingly, each data point may include an AUC value and the corresponding maximum pressure value that occurred during the respective pulse wave. In other examples, the pressure value used to generate each data point may be an average pressure determined for each of the respective normalized pulse waves. Accordingly, each data point may include an AUC value and a corresponding average pressure value determined during from the respective pulse wave. In still other examples, the process 400 may use other methods to generate the data points using AUC values and the pressure signal 404.

Similarly, the process 400 may, additionally or alternatively, generate data points using the determined PAT values and the pressure signal 404. As described herein, generating a data point may include associating a PAT value with a pressure value from the pressure signal 404, or associating a PAT value with a calculated or derived pressure value from the pressure signal 404 (e.g., a corresponding maximum pressure, average pressure, etc.).

At operation 426, the generated data points may be used to determine one or more components of a user's blood pressure. For example, the process 400 may determine a systolic blood pressure, a diastolic blood pressure, a mean arterial pressure (MAP), and/or another measure or component of a user's blood pressure, using the determined data points. Two such methods are described herein with reference to FIGS. 5A-5B and 6A-6B, using data points generated with AUC values and PAT values, respectively.

The process 400 may include, at operation 428, displaying one or more of the determined components of the user's blood pressure. For example, the diastolic and/or systolic blood pressure and/or the mean arterial pressure may be displayed on the device display. In some examples, the systolic and diastolic pressures may be stored in a memory unit of the device (e.g., memory 138), and/or may be stored in network-based storage (e.g., cloud storage).

In some variations, process 400 may be performed in instances where the optical cardiac signal 406 is a blood flow signal, such as when one or more optical sensors of the device (e.g., optical sensor(s) 106) include a laser doppler-based optical sensor capable of providing a blood flow signal. Additionally or alternatively, the optical cardiac signal 406 may include a signal associated with blood flow and/or a signal associated with blood volume. Process 400 may analyze the optical cardiac signal that includes at least the blood flow signal to determine appropriate metric values with which process 400 may generate data points.

In some variations, one or more additional cardiac signals from an additional skin region (or regions) of the user may be obtained as part of process 400. For example, in instances where the pressure signal 404 and the cardiac signal 406 are measured from a finger of the user, an additional cardiac signal may be obtained from a wrist of the user. The additional cardiac signal(s) may be analyzed with the cardiac signal 406 as part of process 400. For example, one or more aspects of a pulse wave may change as the pulse wave propagates into digital arteries, and an additional cardiac signal obtained from the wrist of the user may be used to compensate for these propagation-induced changes that may be present in the cardiac signal 406 obtained from a finger of the user.

In variations in which an additional cardiac signal is obtained as part of process 400, the additional cardiac signal may be measured by the same device that is used to measure the pressure signal 404 and the cardiac signal 406. For example, a single device may include a BP sensing assembly (e.g., BP sensing assembly 105) and an additional sensor (e.g., an additional optical sensor configured to obtain a PPG signal and/or a laser doppler signal from the additional skin region). In other variations, the additional cardiac signal may be measured by a separate device that is used to measure the pressure signal 404 and the cardiac signal 406. For example, a first device may include a BP sensing assembly used to measure the pressure signal 404 and the cardiac signal 406 and a second device may include an optical sensor configured to measure the additional cardiac signal from the additional skin region. In these instances, the second device may transmit the additional cardiac signal to the first device, such that it may be used by the first device as part of process 400.

In some examples, determining metric values may include determining metric values other than (or in addition to) AUC and PAT values associated with normalized pulse waves. In some embodiments, generating data points, such as at operation 423, may include generating multiple sets of data points. For instance, process 400 may generate a first set of data points using the pressure signal 404 and metric values associated with normalized pulse waves associated with a blood volume signal. Process 400 may generate a second set of data points using the pressure signal 404 and metric values associated with a blood flow signal (which may or may not utilize normalized pulse waves of the blood flow signal). Process 400 may use the first and/or second set of data points to determine blood pressure at operation 426.

In some variations, the process 400 may be used to perform multiple blood pressure measurements. In some of these variations, one or more aspects of the process 400 may change between successive measurements. For example, it may be possible that once an initial measurement has been made, information from the initial measurement may be used in one or more subsequent measurements. Accordingly, these subsequent measurements may be performed with a narrower range of pressure values, which may allow for shorter measurement times.

For example, during a first measurement session performed during a first measurement period, the process 400 may include providing guidance/feedback to the user during the first measurement period (such as at operation 420) using a first pressure profile configured to obtain applied pressure measurements that spans a first overall range of target pressures. This first overall range may include an upper pressure boundary that is expected to exceed a systolic pressure of the user and a lower pressure boundary that is expected to be below a diastolic pressure of the user. Accordingly, the first measurement session may generate a first set of data points that is used to determine the user's blood pressure corresponding to the first measurement period.

During a second measurement session performed during a subsequent second measurement period, process 400 may include providing guidance/feedback to the user during the second measurement period using a second pressure profile configured to obtain applied pressure measurements that spans a second overall range of target pressures that is different than the first range of target pressures. For example the second overall range may be narrower than the first overall range. In some instances, the second overall range may include an upper pressure boundary that is expected to be below the systolic pressure of the user and/or may include a lower pressure boundary that is expected to be above the diastolic pressure of the user. The first measurement session may generate a second set of data points that is used to determine the user's blood pressure corresponding to the first measurement period. Although the second measurement session may not yield as wide of a range of data points, information from the first measurement session may be used to compensate for this difference. For example, the first set of data points may be used to establish a relationship between applied pressure and morphologic features of the normalized pulse waves (such as described with respect to FIGS. 5A-5B and 6A-6B). Process 400 may analyze the second set of data points using the relationship established with the first set of data points to determine the blood pressure of the user with the second set of data points.

By utilizing a narrower second range of target pressures, the second measurement period may be shorter than the first measurement period. This may allow for a quicker determination of blood pressure during these subsequent measurements. In some variations, the information from the first measurement session may utilized in subsequent measurement sessions (e.g., one or more iterations of the second measurement session) that are performed within a threshold period of time of the first measurement session. For example, when a relationship between applied pressure and morphologic features of the normalized pulse waves is established, this relationship may be used in any subsequent measurements performed during the threshold period of time. This may allow for relatively shorter measurement durations during these subsequent measurements. The threshold period of time may be on the order of hours, days, or weeks. After the threshold period of time has expired, the first measurement session may be repeated to the reestablish relationship between applied pressure and morphologic features of the normalized pulse waves. Additionally or alternatively, the first measurement session may be repeated on an as-needed basis. For example, one or more characteristics of a subsequent measurement may indicate a need for the first measurement session to be repeated.

FIG. 5A depicts examples of three different normalized pulse waves 504a-504c, such as may be determined by process 400 (e.g., at step 424). The first example normalized pulse wave 504a has a corresponding first AUC 506a, the second example normalized pulse wave 504b has a corresponding second AUC 506b, and the third example normalized pulse wave 504c has a corresponding third AUC 506c. FIG. 5B depicts an AUC graph 502 that includes a set of data points 510, which may be generated by process 400 (e.g., at operation 423). Each of the data points 510 includes an AUC value and corresponding pressure value, which are used to determine systolic and/or diastolic blood pressure, as described herein.

The data points 510 may indicate a relationship between AUC and pressure. For example, during the portion of the measurement when the user is applying lesser amounts of pressure, the compliance of the blood vessels in the finger (and/or other portion of the user's body) may remain fairly constant, or may otherwise not be significantly affected by the applied pressure. Thus, the morphology of each normalized pulse wave may remain relatively unchanged as a result of these lower pressures, which may further result in relatively constant AUC. This effect is illustrated by the relatively flat portion of the AUC graph 502 for applied pressure lower than a first pressure value 508a.

As pressure is increased beyond the first pressure 508a, the compliance of the vessel (and/or other parameters of the vessel) may be affected by the pressure. As a result of this change in compliance (or other parameter), increasing pressure beyond the first pressure 508a, may result in an increase in the AUC of the corresponding normalized pulse waves.

In some examples, the AUC may increase and reach a global (or local) AUC maximum corresponding to the second AUC 506b value, at a second pressure 508b. The second AUC 506b may be identifiable as a first feature of the data points 510 (such as by process 400), and the corresponding second pressure 508b may represent the user's diastolic pressure.

As pressure is increased beyond the second pressure 508b, the increasing pressure may cause further effects on finger vascular compliance. The AUC of each normalized pulse wave may begin to decrease until a third pressure 508c is reached. The third pressure 508c may represent vascular occlusion, where the externally applied pressure exceeds systolic pressure. Further increases in applied pressure beyond the third pressure 508c may cause no further decrease, or negligible decrease, in the AUC beyond the third AUC value 506c. This point on the AUC graph 502 may be identifiable as a second feature of the data points 510, and the corresponding third pressure 508c may be the user's systolic pressure.

Accordingly, to determine a user's diastolic pressure and systolic pressure, the first and second features of the data points 510 may be identified, and the corresponding second pressure 508b and third pressure 508c may be taken as the diastolic and systolic pressures, respectively.

Any of a wide variety of methods may be used to identify the first and second features. For example, one or more first derivative values of the data points 510, one or more second derivative values of the data points 510, and/or a combination of these and/or other values, metrics, and parameters associated with data points 510 may be used to determine the first and second features.

In some embodiments, and as described herein, identifying the first feature may include identifying a data point corresponding to the maximum AUC value. The pressure value associated with this data point (e.g., the second pressure 508b) may be take as the diastolic pressure. In some variations, several pressure values associated with data points near the maximum AUC value may be used to determine the diastolic pressure. For instance, the pressure values from several of the data points 510 may be averaged to determine the diastolic pressure. In other instances, an interpolated pressure may be determined for neighboring data points, and the interpolated pressure may be taken as the diastolic pressure. In still other instances, a line may be fit to the data points 510, and the fit line 518 may be used to determine the diastolic pressure. The fit line 518 may, in some examples, include a series of lines fit to various segments of the data points 510.

The systolic pressure may be similarly determined. Specifically, the second feature may be identified using any of a wide variety of methods. The corresponding pressure may be determined as described herein, such as by averaging the pressure values associated with several of the respective data points 510, performing interpolation, using a fit line 518, etc.

While FIG. 5A depicts examples of normalized pulse waves 504a-c with a generally similar morphology, in other examples, the normalized pulse waves may have morphologies different than those depicted, and different from one another. For instance, the morphology of the normalized pulse waves 504a-c includes two discernible peaks. In other examples, the normalized pulse waves may have greater or fewer peaks, or may have no discernible peaks. In still other examples, the normalized pulse waves may otherwise have different (or significantly different) morphologies.

FIG. 6A depicts an example of how an ECG signal 602 and an optical cardiac signal 606 may be used to calculate PAT. FIG. 6B depicts a PAT graph 601 that includes a set of data points 619 (such as may be generated at operation 423 of process 400). Each of the data points 619 includes a PAT value and corresponding pressure value, which are used to determine diastolic and/or systolic pressure, as described herein.

Referring to FIG. 6A, determining a PAT value includes determining the time between a predetermined portion of the ECG signal 602 and a predetermined portion of the morphology of corresponding pulse wave (or normalized pulse wave) 607 of the optical cardiac signal 606. In the example depicted, an R-wave peak 604 is identified from the ECG signal 602, such as by using any of a variety of established R-wave peak detection methods. The R-wave peak 604 may signify the peak of cardiac contraction, which may initiate the corresponding normalized pulse wave 607.

The base 608 may be considered to be the initiation point of the normalized pulse wave 607. In the example depicted, the base 608 is calculated from features of the pulse wave 607, which may be identified using any of a variety of established pulse wave detection methods. A first point 610 is determined, where the first point 610 corresponds to the first occurrence of a pulse wave minimum following the preceding R-wave peak 604. A first tangent line 612 that is tangent to the first point 610 is then determined. A second point 614 on the normalized pulse wave 607 is determined, where the second point corresponds to the maximum first derivative of the normalized pulse wave 607. A second tangent line 616 that is tangent to the second point 614 is established. The base 608 may then be determined as the intersection of the first tangent line 612 and second tangent line 616. In other examples, the base 608 may be determined using another method.

Using this approach, PAT values may be determined for each of the series of pulse waves (and corresponding R-waves) occurring during the measurement period (such as may be determined at operation 422 of process 400). A set of data points 619 may then be generated (such as at operation 423 of process 400), where each of the data points 619 includes a PAT value and corresponding pressure value.

The data points 619 may indicate a relationship between PAT and pressure. For example, during the portion of the measurement when the user is applying lesser amounts of pressure, the compliance of the blood vessels in the finger may remain fairly constant, or may otherwise not be significantly affected by the applied pressure. Thus, the morphology of each normalized pulse wave may remain relatively unchanged as a result of these lower pressures, which may further result in relatively constant PAT. This effect is illustrated by the relatively flat portion of the PAT graph 601 for applied pressure lower than the first pressure value 624a (e.g., where the PAT remains relatively constant at a first PAT value 622a).

As pressure is increased beyond the first pressure 624A, the compliance of the vessel (and/or other parameters of the vessel) may be affected by the pressure. The PAT may reach a global (or local) minimum at a second PAT value 622b, which corresponds to a second pressure value 624b. The second pressure value 624b may correspond to the user's diastolic BP. The second PAT value 622b may be identifiable as a first feature of the data points 619, and the corresponding second pressure 624b may represent the user's diastolic pressure.

As pressure is increased beyond the second pressure 624b, the increasing pressure may cause further effects on finger vascular compliance. The PAT may begin to increase until a third pressure 624c is reached. The third pressure 624c may represent vascular occlusion, where the externally applied pressure exceeds systolic pressure. Further increases in applied pressure beyond the third pressure 624c may cause no further increases, or negligible increases, in PAT, which may remain constant (or relatively constant) at a third PAT value of 622c. This point on the PAT graph 601 may be identifiable as a second feature of the data points 619, and the corresponding third pressure 624C may be the user's systolic pressure.

Similar to the description provided herein for FIG. 5B, any of a wide variety of methods may be used to identify the first and second features from the data points 619, and to determine the corresponding diastolic and systolic pressures.

Depending on the portion of the body that is measured during a blood pressure measurement, it may be desirable to position a BP sensing assembly to contact a particular location of the user's skin. For example, when a user's finger is used to contact a BP sensing assembly during a blood pressure measurement, some regions of a user's fingertip (e.g., those containing larger arteries) may be better suited for performing a blood pressure measurement as described herein. Accordingly, it may be desirable to identify a target skin location, such as a particular location of a user's finger, that meets certain selection criteria, and to perform a blood pressure measurement using the target skin location.

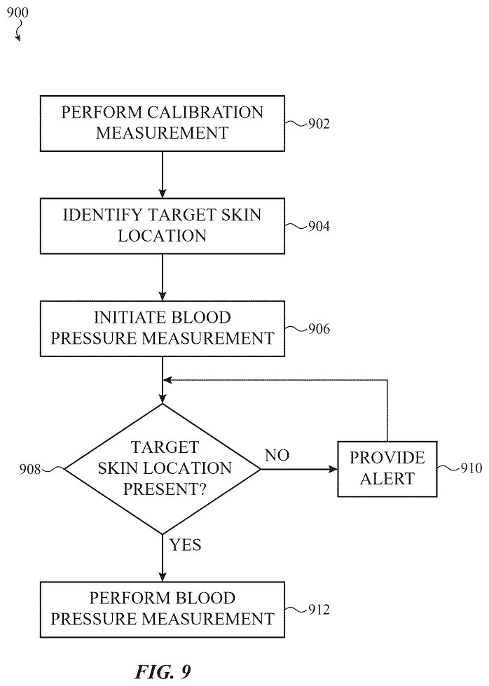

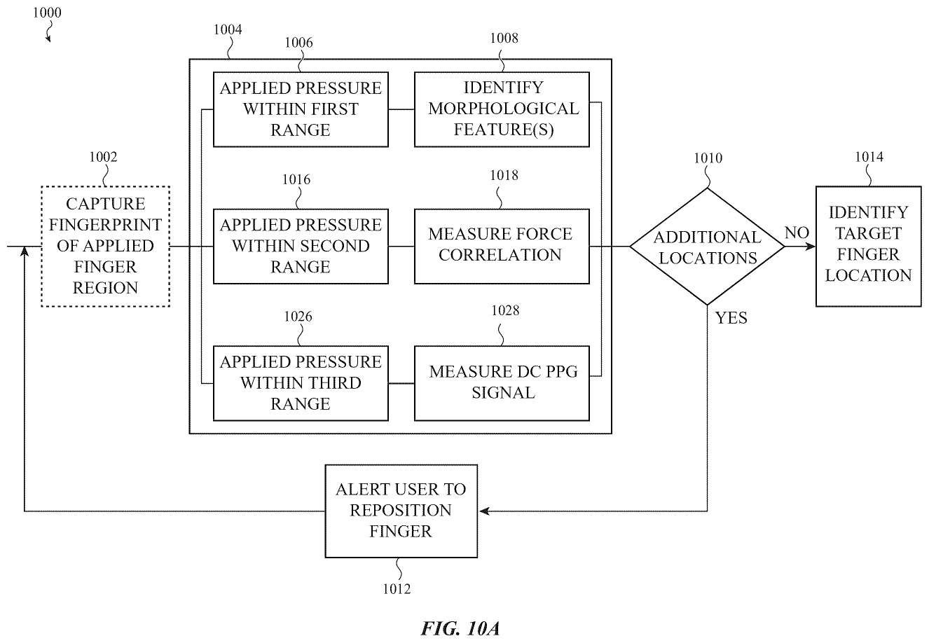

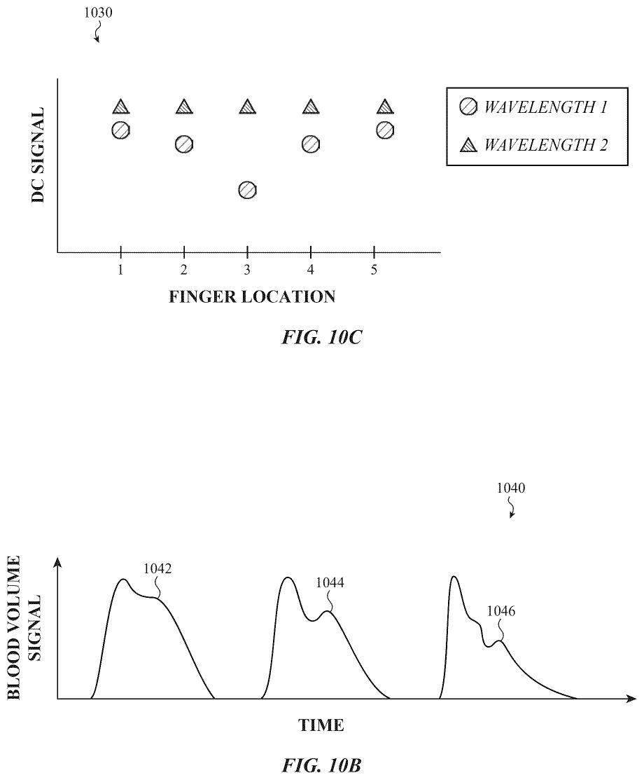

In some variations of the devices described herein, a BP sensing assembly of the device may include a fingerprint sensor that is configured to detect which portion of a user's fingertip is contacting BP sensing assembly (e.g., in contact with an interface surface of the BP sensing assembly) during a blood pressure measurement. In these instances, a target skin location of a user's finger (also referred to herein as a target finger location) may be associated with a corresponding portion of the user's fingerprint, such as described herein with respect to FIGS. 9 and 10A. The fingerprint sensor may be used, such as described herein with respect to FIG. 9, to determine whether the target skin location is contacting the BP sensing assembly during a given blood pressure measurement session.

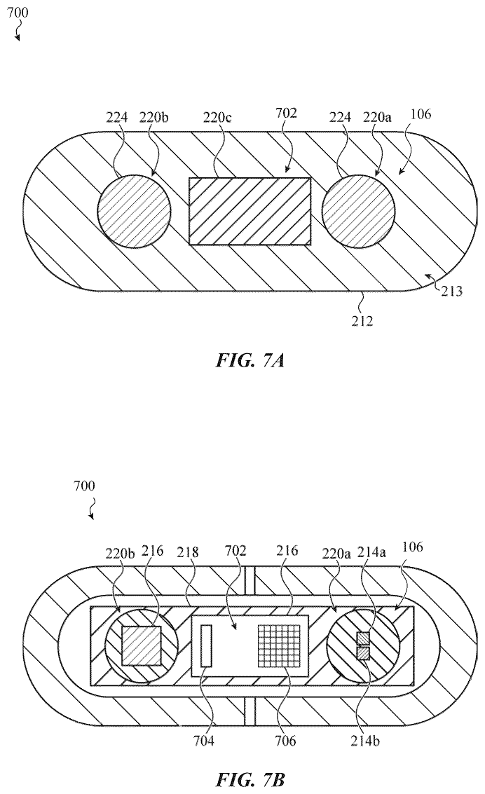

FIG. 7A shows a top view of a variation of a BP sensing assembly 700 that is configured and labeled the same as the BP sensing assembly 105 of FIGS. 2B and 2C, except that the BP sensing assembly 700 includes a fingerprint sensor 702. FIG. 7B shows the BP sensing assembly 700 of FIG. 7A with the sensor housing 212 removed, such that components of the fingerprint sensor 702 and the optical sensor 106 are visible. The fingerprint sensor 702 is configured to determine a portion of a user's fingerprint that is positioned against a corresponding portion of the interface surface 213. For example, the BP sensing assembly 700 may include a third aperture 220c (which may include an optical window material as described herein) and the fingerprint sensor 702 may be configured to determine a portion of a user's fingerprint that is positioned over the third aperture 220c.

The fingerprint sensor 702 may be any type of fingerprint sensor (e.g., a capacitive fingerprint sensor, an optical fingerprint sensor, an ultrasonic fingerprint sensor, or the like) as will readily be understood by one of ordinary skill in the art. In the variation shown in FIGS. 7A and 7B, the fingerprint sensor is configured as an optical fingerprint sensor. Specifically, the fingerprint sensor 702 includes an optical emitter 704 (e.g., a LED or the like) that is operable to illuminate a region of a user's finger through the interface surface 213 (e.g., via the third aperture 220c). The fingerprint sensor 702 further includes a sensor array 706 (e.g., a CMOS image sensor or the like) that is positioned to capture one or more images of the user's finger while it is illuminated by the optical emitter 704. The captured image(s) may be analyzed (e.g., to identify ridges and valleys of the user's finger) to determine which portion of the user's fingerprint is positioned in contact with the interface surface 213.

It should be appreciated that the fingerprint sensor 702 of FIGS. 7A and 7B is just one example, and that a fingerprint sensor may be incorporated into the BP sensing assembly 700 in any suitable manner. For example, while components of the fingerprint sensor 702 (e.g., the light source 704 and the sensor array 706) are shown in FIG. 7B as being mounted to the same PCB 218 as the components of the optical sensor 106, the fingerprint sensor 702 may alternatively be mounted to a separate PCB. Similarly, while the fingerprint sensor 702 is shown in FIG. 7B as including a dedicated optical emitter 704 (e.g., separate from the optical emitters of the optical sensor 106), the BP sensing assembly 700 may be configured such that the fingerprint sensor 702 and the optical sensor 106 share a common optical emitter.

In some variations, a BP sensing assembly as described herein may include an optical sensor that is configured to selectively measure different portions of a skin region that is positioned in contact with the BP sensing assembly. This may allow for the optical sensor to measure a target skin location, even if there is target skin location is not precisely positioned at a desired position relative to the BP sensing assembly.

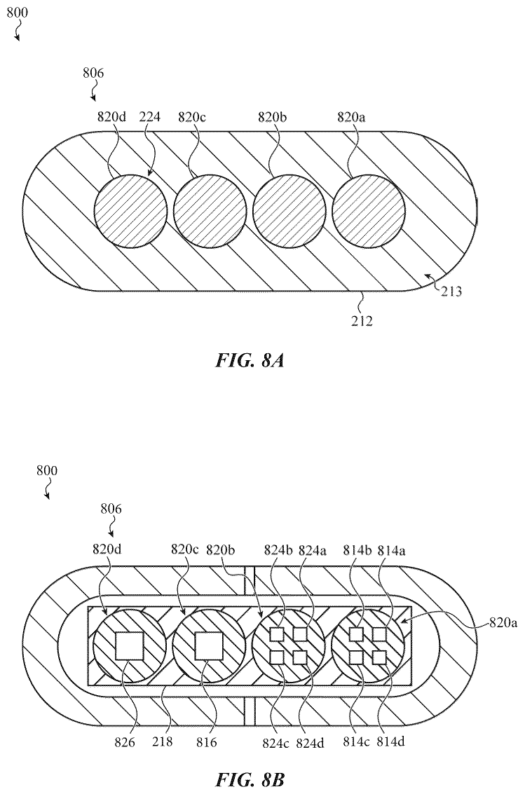

FIG. 8A shows a top view of a variation of a BP sensing assembly 800 that is configured and labeled the same as the BP sensing assembly 105 of FIGS. 2B and 2C, except that the optical sensor 106 has been replaced with optical sensor 806. FIG. 8B shows the BP sensing assembly 800 of FIG. 8A with the sensor housing 212 removed, such that components of the optical sensor 806 are visible. In the variation shown in FIGS. 8A and 8B, the optical sensor 806 includes multiple photodetectors and multiples sets of optical emitters. When the optical sensor 806 is operated to measure a cardiac signal during a blood pressure measurement, the BP sensing assembly 105 may select a photodetector and a set of optical emitters to perform the measurement. This selection may at least partially determine which portion of a user's skin is measured by the optical sensor 806 during the blood pressure measurement.

For example, the optical sensor 806 includes a first set of optical emitters 814a-814d, a second set of optical emitters 824a-824d, a first photodetector 816, and a second photodetector 818. While the first set of optical emitters 814a-814d and a second set of optical emitters 824a-824d are each shown in FIG. 8B as having four optical emitters, it should be appreciated that each set of optical emitters may include more or fewer optical emitters as may be desired. Additionally, while each photodetector and each set of optical emitters is shown in FIGS. 8A and 8B as being positioned within a different corresponding aperture (e.g., the first set of optical emitters 814a-814d is positioned in a first aperture 820a, the second set of optical emitters 824a-824d is positioned in a second aperture 820b, the first photodetector 816 is positioned in a third aperture 820c, and the second detector 826 is positioned in a fourth aperture 820d), it should be appreciated that some or all of these photodetectors and emitters may be positioned in a common aperture.

During operation of the BP sensing assembly 800, the optical sensor 806 may perform cardiac signal measurements using one of the multiple photodetectors and one of the multiples sets of optical emitters. For example, when a skin region is positioned against the BP sensing assembly 800 (e.g., against the interface surface 213), a cardiac signal measurement performed using the first set of optical emitters 814a-814d (or a subset thereof) and the first photodetector 816 may measure a different portion of the skin region than a cardiac signal measurement performed by the second set of optical emitters 824a-824d (or a subset thereof) and the second photodetector 826. Accordingly, the photodetector and emitter(s) used to perform a cardiac signal measurement may be selected to more closely measure a target skin location of the user.

In some instances, a target skin location may be identified prior to performing a given blood pressure measurement. For example, FIG. 9 shows a process 900 for performing a blood pressure measurement at a target finger location. The example process 900 may be performed by one or more processors as described herein (e.g., processor 136). Process 900 may be performed as a method, or may be stored as instructions on a non-transitory computer-readable storage device, such that the processor may utilize these instructions to perform the various steps of the processes described herein. Further, the processor may be operatively coupled to a memory (e.g., memory 138), where the processor is configured to execute instructions that cause the processor to perform process 900.

At step 902, prior to initiating a blood pressure measurement, the process 900 includes performing a calibration measurement. The calibration measurement may be performed using the same device that will be used to perform the subsequent blood pressure measurement, or may be performed using a different device (e.g., the calibration measurement may be performed by a first device and the results of the calibration measurement may be sent to a second device that performs the blood pressure measurement). In variations where the calibration measurement is performed using the same device that performs the blood pressure measurement, the calibration measurement may be performed using the same BP sensing assembly that performs the blood pressure measurement, or may be performed using a different BP sensing assembly or other set of sensors of the same device.

At step 904, the process 900 includes identifying a target skin location from the calibration measurement. In instances where a finger is used to perform a blood pressure measurement, the target skin location may be a target finger location. The calibration measurement may identify a target skin location as a portion of a user's skin that meets a set of selection criteria, such as described herein with respect to FIGS. 10A-10C. While the processes described herein with respect to FIGS. 9 and 10A-10C are described herein with respect to a single target skin location, it should be appreciated that the processes described herein may be used to identify multiple target skin locations that meet the set of selection criteria.

At step 906, the process 900 includes initiating a blood pressure measurement. As part of initiating the blood pressure measurement, the process 900 includes, at step 908, determining whether a target skin location (a particular target skin location or any of a plurality of target skin locations identified during one or more calibration measurements) is positioned against the BP sensing assembly that is used to perform the blood pressure measurement. For example, in variations in which the BP sensing assembly includes a fingerprint sensor (e.g., fingerprint sensor 702), the fingerprint sensor may be used to determine whether the target skin location is positioned against the BP sensing assembly. In these variations, the target skin location may be determined to be present when the fingerprint sensor determines that a portion of a user's fingerprint corresponding to the target skin location is positioned in contact with an interface surface of the BP sensing assembly.

In some variations, determining whether the target skin location is positioned against the BP sensing assembly at step 908 also includes determining whether the target skin location is positioned against the BP sensing assembly with a target orientation. For example, it may be desirable for a user to position their finger against a BP sensing assembly while oriented in a certain direction. In variation in which the BP sensing assembly includes a fingerprint sensor (e.g., fingerprint sensor 702), the fingerprint sensor may also be used to determine (using a corresponding orientation of the detected fingerprint) whether the target skin location is positioned with the target orientation.

If, at step 908, the process determines that the target skin location is not properly positioned against the BP sensing assembly (e.g., is not present or is positioned with an improper orientation relative to the BP sensing assembly), the process 900 includes providing an alert at step 910. The alert (which may include any combination of visual feedback, haptic feedback and/or audio feedback) may prompt a user to reposition the skin region that is positioned against the BP sensing assembly. The process may return to step 908 and again determine whether the target skin location is properly positioned against the BP sensing assembly.

If, at step 908, the process 900 determines that the target skin location is properly positioned against the BP sensing assembly, the process may include performing the blood pressure measurement at step 912. The blood pressure measurement may be performed in any manner as described herein, such as with respect to the process 400 of FIG. 4.

The devices described herein may be configured to perform the calibration measurement of step 902 at different intervals. In some instances, the calibration measurement may be performed once (e.g., as part of an initial onboarding experience) and the target skin location(s) identified from the calibration measurement may be used for all subsequent blood pressure measurements performed by the device. In other instances, the calibration measurement may be repeated on a periodic basis (e.g., once a week, once a month, or the like). In these instances, the results of a given calibration measurement may augment (e.g., by identifying new target skin locations) and/or replace (e.g., by replacing previously-identified target skin locations) the results of a previously-performed calibration measurement. In still other instances, a new calibration measurement may be performed before each blood pressure measurement.

FIG. 10A shows an example process 1000 by which one or more target finger locations may be identified using a calibration measurement. The example process 1000 may be performed by one or more processors as described herein (e.g., processor 136). Process 1000 may be performed as a method, or may be stored as instructions on a non-transitory computer-readable storage device, such that the processor may utilize these instructions to perform the various steps of the processes described herein. Further, the processor may be operatively coupled to a memory (e.g., memory 138), where the processor is configured to execute instructions that cause the processor to perform process 1000.

While the process 1000 is described herein with respect to a user's finger, it should be appreciated that this process may be used to identify a target skin location with respect to other skin regions as may be desired. During the calibration measurement, the process 1000 includes performing, for each of a plurality of candidate finger locations, a characterization measurement to generate one or more metrics associated with that finger location. After performing a plurality of characterization measurements corresponding to the plurality of candidate finger locations, the process 1000 may identify one or more target finger locations from the plurality of candidate finger locations. Specifically, the results of the characterization measurements (e.g., the metric(s) associated with each candidate finger location) may be analyzed to determine whether any of the candidate finger locations meets a set of selection criteria.

Specifically, a user may initially position a first region of their finger against a BP sensing assembly as described herein. This first region may define a first candidate finger location. In variations in which the BP sensing assembly includes a fingerprint sensor (e.g., fingerprint sensor 702), the process 1000 may include capturing a corresponding fingerprint for each candidate finger region at step 1002. For example, when the first region corresponding to the first candidate finger location is positioned in contact with the BP sensing assembly, the fingerprint sensor may capture a first fingerprint associated with the first candidate finger location at step 1002.

For each candidate finger location, the process 1000 includes performing a characterization measurement at step 1004 that includes one or more individual measurements. Each of these individual measurements may be associated with a corresponding pressure range, such that the individual measurement is performed while the user is applying a pressure that falls within the corresponding pressure range. While the characterization measurement of FIG. 10A includes three individual measurements, it should be appreciated that a characterization measurement may include more or fewer individual measurements as may be desired.

In some variations, the characterization measurement may include a first individual measurement in which i) BP sensing assembly captures a cardiac signal from the applied finger region, and ii) identify one or more morphological features in one or more pulse waves of the captured cardiac signal. The presence and/or characteristics of the morphological feature(s) may be part of the metric(s) generated for that candidate finger location. For example, FIG. 10C shows a graph 1040 of a blood volume signal (which may represent the cardiac signal captured by the BP sensing assembly) over time. The graph 1040 includes three different pulse waves (e.g., a first pulse wave 1042, a second pulse wave 1044, and a third pulse wave 1046). The first pulse wave 1042 may represent a cardiac signal captured from a first finger location in which predominantly capillaries are present in the tissue measured by the BP sensing assembly, the second pulse wave 1044 may represent the cardiac signal captured from a second finger location in which relatively more arterioles are present, and the third pulse wave 1046 may represent the cardiac signal captured from a third finger location in which a larger artery is present.

The morphological features of these pulse waves may depend at least in part on the relative size of the blood vessels that are measured during the cardiac signal measurement. For example, the morphological features generally become sharper as larger blood vessels are measured in the cardiac signal. For example, one or more characteristics of the dicrotic notch may vary with finger location. Accordingly, by analyzing one or more morphological features associated with the pulse waves of the cardiac signal, the process 1000 may determine whether a particular finger location is more likely to be associated with larger blood vessels.

When performing instances of the first individual measurement across different candidate finger positions, it may be desirable to maintain a pressure applied by the user's finger within a first range. For example, at step 1006, the process 1000 may include determining that a pressure applied by the candidate finger location is within the first range. At step 1008, the process may include performing the first individual measurement while the applied pressure is within the first range to capture a cardiac signal, and may include identifying one or more morphological features in one or more pulse waves of the cardiac signal.

At step 1010, the process 1000 may include determining whether additional candidate finger locations should be measured using a corresponding characterization measurement. For example, it may be desirable for the calibration measurement to evaluate at least a minimum number of candidate finger locations. In some variations, additional candidate finger locations may be analyzed until the process 1000 is able to identify a target finger location that meets a set of selection criteria. If the process 1000 determines at step 1010 that additional candidate finger locations should be measured, the process 1000 may include prompting a user to reposition their finger at step 1012. This prompt may include an alert or notification (e.g., using visual feedback, haptic feedback, audio feedback, combinations thereof, or the like) that directs a user to reposition their finger such that a new finger region is in contact with the BP sensing assembly (and thereby defines a new candidate finger location). The process 1000 may return to step 1002, where a new fingerprint is obtained for the new candidate finger location and/or a new characterization measurement is performed for the new candidate finger location. This process 1000 may be repeated as needed until it is determined, at step 1010, that no additional candidate finger locations should be measured.

At step 1014, the process 1000 includes identifying one or more target finger locations from the plurality of candidate finger locations. For example, the process 1000 may analyze the results of the plurality of characterization measurements (e.g., corresponding to the plurality of candidate finger locations) to determine which candidate finger locations satisfy the set of selection criteria. For example, the results of the first individual measurements at step 1008 may be analyzed to determine which candidate finger locations exhibited certain morphological features indicative of the presence of larger blood vessels. If multiple candidate finger locations satisfy the set of selection criteria, some or all of these candidate finger locations may be identified as target finger locations. In some variations, each candidate finger location that satisfies the set of selection criteria is identified as a target finger location. In other variations, each of a subset of candidate fingers that satisfy the set of selection criteria is identified as a target finger location.

In some variations, the characterization measurement may (in addition to or as an alternative to the first individual measurement performed at step 1008) include a second individual measurement in which a pressure signal measured by the BP sensing assembly (e.g., via pressure sensor 108) is correlated with a PPG signal measured by the BP sensing assembly (e.g., via optical sensor 106). Specifically, the pressure signal and the PPG signal may each be separated into a corresponding static portion (DC signal component) and a corresponding time-varying portion (AC signal component), and the AC signal component of the pressure signal may be correlated with the AC signal component of the PPG signal. For example, if the pressure applied a candidate finger location is at or near a user's mean arterial pressure, a higher correlation between the AC signal components of the pressure and PPG signals may be indicative of the presence of larger blood vessels within the candidate finger location.

When performing instances of the second individual measurement across different candidate finger positions, it may be desirable to maintain a pressure applied by the user's finger within a second range. For example, at step 1016, the process 1000 may include determining that a pressure applied by applied by the candidate finger location is within the second range. In some variations, the second range may be selected such that applied pressure is likely to be at or near the user's mean arterial pressure. At step 1018, the process 1000 may include performing the second individual measurement for the candidate finger location while the applied pressure is within the second range to determine an amount of correlation between the AC signal components of the pressure signal and the PPG signal. The correlation amount may be part of the metric(s) generated for that candidate finger location, and may be analyzed in determining whether the candidate finger location satisfies the set of selection criteria.

In some variations, the characterization measurement may (in addition to or as an alternative to the first individual measurement and/or the second individual measurement) include a third individual measurement in which i) one or more PPG signals are measured, and ii) a corresponding DC signal component of each PPG signals is determined. For example, if the pressure applied by a candidate finger location is above a user's systolic blood pressure, certain wavelengths of light (e.g., green light) will be more strongly absorbed by a user's finger near larger blood vessels. For example, FIG. 10C shows a chart 1030 of the DC signal component of measured PPG signal values as a function of i) candidate finger position and ii) wavelength. Specifically, chart 1030 includes the DC signal component magnitudes of PPG signals obtained at a first wavelength (“wavelength 1”) for five different candidate finger locations (labeled 1-5). Candidate finger location 3 may represent a finger location that includes a larger artery, and thus may have the lowest DC signal component of the five candidate finger locations for the first wavelength.

Accordingly, the third individual measurement may include i) obtaining (e.g., using an optical sensor of a BP sensing assembly) a first PPG signal of a first wavelength from the candidate finger location and ii) determining a DC signal component of the first PPG signal. In some variations, the first wavelength is a green wavelength. At step 1026, the process 1000 may include determining that a pressure applied by applied by the candidate finger location is within a third range. In some variations, the third range may be selected such that applied pressure is likely to be above a user's systolic pressure. At step 1028, the process 1000 may include performing the third individual measurement for the candidate finger location while the applied pressure is within the third range to determine a DC signal component of the first PPG signal. The DC component of the first PPG signal may be part of the metric(s) generated for that candidate finger location, and may be analyzed in determining whether the candidate finger location satisfies the set of selection criteria.

The DC component of the first PPG signal may also depend at least in part on the amount of ambient light that is present when obtaining the first PPG signal. In some instances, may be difficult to identify whether a change in the DC signal component of the first PPG signal between different finger locations is a result of changes in the measured vasculature or a change in ambient light. Accordingly, it may be desirable for the third individual measurement to collect one or more additional PPG signals at other wavelengths. The absorption of other wavelengths of light (e.g., red or infrared wavelengths) may be less dependent on the presence or absence of larger blood vessels. For example, chart 1030 includes the DC signal component magnitude of PPG signals obtained at a second wavelength (“wavelength 2”) at the five different candidate finger locations. The DC signal components for these PPG signals may not vary significantly across the finger locations, as compared with the first wavelength, even with the presence of a larger artery at finger location 3.

Accordingly, in some variations the third individual measurement may also include i) obtaining (e.g., using an optical sensor of a BP sensing assembly) a second PPG signal of a second wavelength from the candidate finger location and ii) determining a DC signal component of the second PPG signal. In some variations, the second wavelength is a red wavelength or an infrared wavelength. The DC component of the second PPG signal may be part of the metric(s) generated for that candidate finger location, and may be analyzed in determining whether the candidate finger location satisfies the set of selection criteria. For example, the DC signal component of the second PPG signal may be taken into account when analyzing the DC signal component of the first PPG signal, which may reduce the impact of changes in the amount of ambient light measured at different candidate finger locations.

In some embodiments, a blood pressure measurement method as described herein (such as process 400), is performed at a first computer system (as described herein) via a system process (e.g., an operating system process, a server system process) that is different from one or more applications executing and/or installed on the first computer system.

In some embodiments, the method is performed at a first computer system (as described herein) via a system process (e.g., an operating system process, a server system process) that is different from one or more applications executing and/or installed on the first computer system.

In some embodiments, the method is performed at a first computer system (as described herein) by an application that is different from a system process. In some embodiments, the instructions of the application, when executed, control the first computer system to perform the method by calling an application programming interface (API) provided by the system process. In some embodiments, the application performs at least a portion of the method without calling the API.

In some embodiments, the application can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application.

In some embodiments, the application is an application that is pre-installed on the first computer system at purchase (e.g., a first party application). In other embodiments, the application is an application that is provided to the first computer system via an operating system update file (e.g., a first party application). In other embodiments, the application is an application that is provided via an application store. In some implementations, the application store is pre-installed on the first computer system at purchase (e.g., a first party application store) and allows download of one or more applications. In some embodiments, the application store is a third-party application store (e.g., an application store that is provided by another device, downloaded via a network, and/or read from a storage device). In some embodiments, the application is a third-party application (e.g., an app that is provided by an application store, downloaded via a network, and/or read from a storage device). In some embodiments, the application controls the first computer system to perform the method by calling an application programming interface (API) provided by the system process using one or more parameters.

In some embodiments, at least one API is a software module (e.g., a collection of computer-readable instructions) that provides an interface that allows a different set of instructions (e.g., API calling instructions) to access and use one or more functions, methods, procedures, data structures, classes, and/or other services provided by a set of implementation instructions of the system process. The API can define one or more parameters that are passed between the API calling instructions and the implementation instructions.

In some embodiments, the set of implementation instructions is a system software module (e.g., a collection of computer-readable instructions) that is constructed to perform an operation in response to receiving an API call via the API. In some embodiments, the set of implementation instructions is constructed to provide an API response (via the API) as a result of processing an API call. In some embodiments, the set of implementation instructions is included in the device that runs the application. In some embodiments, the set of implementation instructions is included in an electronic device that is separate from the device that runs the application.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining a metric, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, the output result may be provided based on non-personal information data or a bare minimum amount of personal information, such as events or states at the device associated with a user, other non-personal information, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A method for determining blood pressure, comprising:
measuring a pressure signal corresponding to a pressure applied by a skin region of a user over a measurement period;
measuring a cardiac signal from the skin region during the measurement period;
identifying a series of pulse waves within the cardiac signal;
determining a series of metric values, wherein each metric value of the series of metric values is associated with a morphology of a respective pulse wave of the series of pulse waves;
generating a series of data points, each data point comprising:
a pressure value from the pressure signal; and
a corresponding metric value from the series of metric values; and
determining, based on the series of data points, a systolic pressure.

2. The method of claim 1, wherein the wherein the cardiac signal is obtained using an optical sensor.

3. The method of claim 1 comprising:
determining, based on the series of data points, a diastolic pressure, wherein:
determining the diastolic pressure comprises:
identifying a first feature of the series of data points; and
determining the diastolic pressure based on a pressure associated with the identified first feature;
and determining the systolic pressure comprises:
identifying a second feature of the series of data points; and
determining the systolic pressure based on a pressure associated with the identified second feature.

4. The method of claim 1, wherein each metric value of the series of metric values is an area under the curve calculated for a corresponding pulse wave of the respective pulse wave of the series of pulse waves.

5. The method of claim 1, wherein each metric value of the series of metric values is a pulse arrival time associated with a respective pulse wave of the series of pulse waves.

6. The method of claim 1, wherein the cardiac signal represents changes in blood volume.

7. The method of claim 1, comprising normalizing the series of pulse waves.

8. The method of claim 2, wherein the optical sensor includes at least a photoplethysmography-based sensor.

9. The method of claim 8, wherein the optical sensor further includes a laser doppler-based optical sensor.

10. A method for determining blood pressure, comprising:
simultaneously measuring, over a measurement period:
a pressure applied by a skin region of a user;
a cardiac signal of the skin region over the measurement period using an optical sensor;
identifying a series of pulse waves within the cardiac signal;
generating a set of data points, each data point comprising:
a pressure value selected based on a portion of the measured skin region pressure corresponding to a respective pulse wave of the series of pulse waves; and
a value of a metric associated with a morphology of the respective pulse wave of the series of pulse waves;
determining, based on the set of data points, a diastolic pressure;
determining, based on the set of data points, a systolic pressure; and
presenting at least one of the diastolic pressure and systolic pressure.

11. The method of claim 10, wherein the optical sensor is a photoplethysmography sensor.

12. The method of claim 10, wherein the metric is an area under the curve calculated for a respective pulse wave of the series of pulse waves.

13. The method of claim 10, wherein the metric is a pulse arrival time associated with a respective pulse wave of the series of pulse waves.

14. The method of claim 10, wherein the cardiac signal represents changes in blood volume.

15. The method of claim 10, wherein the pressure is applied to display configured to measure the applied pressure.

16. The method of claim 11, wherein the optical sensor further includes a laser doppler-based optical sensor.

17. A device, comprising:
a set of sensors;
a memory; and
one or more processors operatively coupled to the memory, wherein the one or more processors are configured to execute instructions causing the one or more processors to:
receive a pressure signal from the set of sensors, the pressure signal corresponding to a pressure applied by a skin region of a user over a measurement period;
receive a cardiac signal from the set of sensors, the cardiac signal corresponding to a blood volume of the skin region during the measurement period;
identify a series of pulse waves within the cardiac signal;
determine a series of metric values, wherein each metric value of the series of metric values is associated with a morphology of a respective pulse wave of the series of pulse waves;
generate a series of data points, each data point comprising:
a pressure value from the pressure signal; and
a corresponding metric value from the series of metric values;
determine, based on the series of data points, a diastolic pressure; and
determine, based on the series of data points, a systolic pressure.

18. The device of claim 17, wherein the set of sensors includes at least a photoplethysmography optical sensor configured to measure the blood volume signal.

19. The device of claim 17, wherein the one or more processors are configured to normalize the series of pulse waves.

20. The device of claim 17, further configured to:
identify a first feature of the series of data points;
determine the diastolic pressure based on a pressure associated with the identified first feature;
identify a second feature of the series of data points;
determine the systolic pressure based on a pressure associated with the identified second feature.
